(19) United States
(12) Patent Application Publication
(10) Pub. No.: US 2014/0100216 A1
(43) Pub. Date: Apr. 10, 2014

(54) STIMULATORS OF INCRETIN HORMONES SECRETION, METHOD FOR PREPARATION AND USE THEREOF

(71) Applicants: Nikolay Filippovich Savchuk, Moscow (RU); Sergey Yevgenievich Tkachenko, San Diego, CA (US); Denis Yevgenievich Nazarenkov, Del Mar, CA (US)

(72) Inventors: Nikolay Filippovich Savchuk, Moscow (RU); Sergey Yevgenievich Tkachenko, San Diego, CA (US); Denis Yevgenievich Nazarenkov, Del Mar, CA (US)

(21) Appl. No.: 14/118,895

(22) PCT Filed: May 12, 2012
(86) PCT No.: PCT/RU2012/000374

§ 371 Date: Nov. 20, 2013

(30) Foreign Application Priority Data
May 20, 2011 (RU) 2011120084

(51) Int. Cl. C07D 267/14; C07D 413/14; C07D 498/04; C07D 413/12; A61K 31/553; A61K 45/06

(52) U.S. Cl. CPC C07D 267/14; A61K 31/553; A61K 45/06; C07D 498/04; C07D 413/12; C07D 413/14
USPC 514/211.05; 540/490

(57) ABSTRACT

The invention relates to the area of medicinal chemistry, pharmacology and medicine and includes description of pharmaceutical compositions and combined medicaments on the base of secretion stimulators and protectors of incretin hormones for treatment of metabolic diseases (among them, diabetes, obesity, metabolic syndrome and the like). The invention consists in that that pharmaceutical composition or combined medicament comprises a derivative of tetrahydrobenzo[f][1,4]oxazepine—either nonsteroidal agonist of bile aids receptor TGR5, or one of endogenous bile acids which stimulate incretin hormones secretion, and also one of the known inhibitors of DPP-IV proteinase. In this case administration of TGR5 agonists is carried out peroral, and administration of endogenous bile acids is exercised rectal in the form of suppository or gel. As proteinase DPP-IV inhibitors could be used Vildagliptin, Saxagliptin, Sitagliptin, Teneligliptin, Linagliptin, Dutogliptin, Alogliptin, Gemigliptin, Carmegliptin and the like. Besides, the invention includes description of novel tetrahydrobenzo[f][1,4]oxazepine derivatives—nonsteroidal agonist of bile aids receptors TGR5, and also methods for their preparation. The invention provides enhancement of therapy effectiveness owing to synergetic action of the components, thus making possible simultaneous treatment of diabetes, and obesity, other metabolic diseases and their cardiovascular and renal complications.

### CROSS REFERENCE TO RELATED APPLICATIONS

This application is a National stage of International application PCT/RU2012/000374 filed May 12, 2012, which claims benefit of foreign priority to the Russian Federation application RU 2011120084 of May 20, 2011. The priority applications are hereby incorporated by reference in their entirety.

### FIELD OF THE INVENTION

The present invention relates to novel physiologically active compounds, novel stimulants of incretin hormones secretion, active components for pharmaceutical composition, to pharmaceutical compositions and combination products comprising either nonsteroidal agonist of bile acids receptor TGR5, or one of endogenous bile acids, which stimulates incretin hormones secretion and also one of the known proteinase DPP-IV inhibitors, and to the combined method for treatment of metabolic, cardiovascular and neurodegenerative diseases, such as diabetes, obesity, metabolic syndrome and the like.

### PRIOR ART

Diabetes together with overweight (obesity or adiposis) is the cause of death for more then 280 thousands of people in USA every year. Recently a new scientific-medicinal term “Diabesity” (the Russian version is “Diabetuchnost”) has appeared, meaning the combination of two interrelated pathologies: diabetes and obesity. Now diabesity is regarded as one of the most serious health problem of mankind, which has become, practically, the first noninfectious epidemic [Schmidt M I, Duncan B B, Diabesity: an inflammatory metabolic condition. Clin Chem Lab Med. 2003; 41: 1120-1130], [Dang M N, Hashem B E-S. The epidemiology of obesity. Gastroenterol Clin North Am. 2010; 39: 1-7].

Diabetes in combination with obesity has become a problem and begun to extend rapidly during the last century. The main cause of the epidemic is considered to be sedentary lifestyle and unhealthy diet [Zimmet P, Alberti K G, Shaw J. Global and societal implications of the diabetes epidemic. Nature 2001; 414: 782-787], and also genetic predisposition [Ling C, Groop L. Epigenetics: a molecular link between environmental factors and type 2 diabetes. Diabetes 2009; 58: 2718-2725]. Diabesity is also an integral component of metabolic syndrome. Expanding of diabetes is taking place with an increasing pace. In 2010 diabetes was registered for 6.4% of world population, prediction for 2030 is 7.7%. [Farag Y, Gaballa M, Diabesity: an overview of a rising epidemic. Nephrol Dial Transplant 2011; 26: 28-35].

At present, special diets—the easiest way for diabesity treatment—do not settle the problem—98% of persons having lost the weight get it back with excess within 5 years. Observable effect has various surgical operations (bariatric surgery) in the fight against diabesity, however they are associated with a large number of side effects connected with the disturbance of hormonal homeostasis [Tharakan G, Tan T, Bloom S. Emerging therapies in the treatment of ‘diabesity’: beyond GLP-1. Trend Pharm Sci. 2011; 32: 8-15].

Up to now numerous efforts of pharmaceutical companies to develop highly effective remedies for diabetes (diabesity) treatment have not led to a notable success. In spite of the fact, that more then 130 novel drug candidates undergo various trials now, only about 10 compounds are proposed to clinical use. Clinical trials of a great number of drug candidates were broken off because of their significant side effects. Thus, for example, the most promising antidiabetic drug “Avandia” (Rosiglitazone— [symbol] PPARgamma receptor agonist), as it happened, increases the risk of heart stroke and cardiac arrest [Thomson Reuters Drug News. (formerly, DailyDrugNews.com) Sep. 28, 2010].

The main reasons for a long succession of failures of researchers consist in insufficient understanding of the most complicated picture of the disease, in particular, existence of numerous signaling systems. Influence on one effector target only leads to escape of pathological process as a result of activation of other mechanisms.

That is why now the main direction in developing antidiabetic remedies is searching for pharmacological agents stimulating important processes capable to inhibit pathology growth (for example, incretin protein GLP1, YYP, GIP secretion) in particular, nonsteroidal agonists of bile acids (BA), which specifically bind to BA receptors and stimulate secretion of incretin hormones (GLP1, YYP, GIP).

The most preferable agonists are agonists of BA receptors TGR5. TGR5 receptors (also known as GPBAR1, M-BAR, AXOR 109 and GPCR19) are GPCR-receptors bound to Gs-proteins [Tiwari, A.; Maiti, P. Drug Disc. Today 2009, 14, 523]. They are mainly expressed in gastrointestinal tract, gall-bladder, spleen, lungs and placenta [Maruyama, T.; Miyamoto, Y.; Nakamura, T.; Tamai, Y.; Okada, H.; Sugiyama, E.; Nakamura, T.; Itadani, H.; Tanaka, K. Biochem. Biophys. Res. Commun. 2002, 298, 714]. BA binding to TGR5 receptors activates adenylate cyclase that results in increasing intracellular cAMP concentration with subsequent activation of MAP-kinase signaling cascade. On the other hand, stimulation of TGR5 receptors regulates a number of metabolic processes [Groen, A. J. J. Hepatol. 2006, 45, 337]. Among other things, it was shown that agonist effect on these receptors of enteroendocrine STC-1 cells increases secretion of GLP-1 peptide substantially. [Katasuma, S.; Hirasawa, A.; Tsujimoto, G. Biochem. Biophys. Res. Commun. 2005, 329, 386]. GLP-1 peptide improves glucose homeostasis in several ways including stimulation of insulin secretion and suppression of glucagon secretion, which is exceptionally important in the treatment of such diseases as diabetes, diabesity and obesity. However, as endogenous BA because of their steroid nature interact simultaneously with several receptors [Makishima, M.; Okamoto, A. Y.; Repa, J. J.; Tu, H.; Learned, R. M.; Luk, A.; Hull, M. V.; Lustig, K. D.; Mangelsdorf, D. J.; Shanz, B. Science 1999, 284, 1362], searching of selective agonists of TGR5 receptors ought to be carried out among small molecules of nonsteroid nature.

Authors of the invention have accomplished synthesis of various heterocyclic compounds and screening of them in experiments with cells of HEK-293 line with expressed by human TGR5 (hTGR5) receptor. Agonistic activity of the compounds was estimated by increasing of intracellular cAMP concentration. As a result, a series of novel compounds exhibiting pronounced agonistic activity towards hTGR5 receptors was found.

Novel derivatives of tetrahydrobenzo[f][1,4]oxazepines in the form of bases and pharmacologically acceptable salts and hydrates were found, they are nonsteroidal agonists of bile acids TGR5 receptors and exhibit properties of incretin hormones stimulator.

Moreover, it has been shown, that physiological effect of novel compounds could be significantly increased if they are used together with DPP-IV proteinase inhibitors simultaneously. Most probably, multidirectional impact of nonsteroidal agonists of bile acids TGR5 and proteinase inhibitors consists in combined simultaneous influence on two metabolic processes. For one thing, this action involves stimulating incretin secretion at the action on bile acids (BA) receptors TGR5 in L-cells by their synthetic nonsteroidal agonists or their natural ligands bile acids (BA). For another thing, their action consists in inhibition of ferments splitting incretin peptides, particularly DPP-IV peptidase.

It was found that the place of combined treatment administration is of special importance.

Novel pharmaceutical compositions and combined medicaments were prepared which comprised nonsteroidal agonist of bile acids receptors TGR5, and/or one of endogenous bile acids or a mixture of endogenous bile acids which stimulate incretin hormones secretion, and also one of known proteinase DPP-IV inhibitors. At this, administration of TGR5 agonists is carried out peroral, and administration of endogenous bile acids—rectally in the form of suppository or gel.

Vildagliptin, Saxagliptin, Sitagliptin, Teneligliptin, Linagliptin, Dutogliptin, Alogliptin, Gemigliptin, Carmegliptin and the like could be used as an DPP-IV proteinase inhibitor.

Synergetic effect of components increases efficiency of therapy considerably, as a result of it simultaneous therapy of diabetes and obesity and other metabolic diseases and their cardiovascular and renal complications becomes possible. The invention provides high efficiency of treatment and simplifies direct care at a great number of patients.

Up to now in the scientific literature (patents, articles, scientific conference abstracts and the like) there was no description of the combined employment of agonists of BA TGR5 receptors and DPP-IV inhibitors. There is also no description of rectal administration of endogenous bile acids in the form of the corresponding drug formulation for metabolic diseases treatment.

### DISCLOSURE OF THE INVENTION

In context of the invention, terms are generally defined as follows:

“Agonists” mean compounds which being bound to receptors of definite type actively promote transferring their specific signal and by that cause biological response of cell.

“Azaheterocycle” means an aromatic or saturated mono- or polycyclic system comprising at least one N-atom in the cycle. Azaheterocycle may have one or more “cyclic system substituents”.

“Active component” (drug-substance) means a physiologically active compound of synthetic or other (biotechnological, vegetable, animal, microbe and so on) origins exhibiting pharmacological activity which is an active ingredient of pharmaceutical composition employing in production and preparation of medicaments.

“Alkyl” means an aliphatic hydrocarbon straight or branched chain with 1-12 carbon atoms. Branched means alkyl chain with one or more “lower alkyl” substituents. Alkyl group may have one or more substituents of the same or different structure (“alkyl substituent”) including halogen, alkenyloxy, cycloalkyl, aryl, heteroaryl, heterocyclyl, aroyl, cyano, hydroxy, alkoxy, carboxy, alkynyloxy, aralkoxy, aryloxy, aryloxycarbonyl, alkylthio, heteroarylthio, aralkylthio, arylsulfonyl, alkylsulfonyl, heteroaralkyloxy, annelated heteroarylcycloalkenyl, annelated heteroarylcycloalkyl, annelated heteroarylheterocyclenyl, annelated heteroarylheterocyclyl, annelated arylcycloalkenyl, annelated arylcycloalkyl, annelated arylheterocyclenyl, annelated arylheterocyclyl, alkoxycarbonyl, aralkoxycarbonyl, heteroaralkyloxycarbonyl or Rₖ^{a}Rₖ₊₁^{a}N—, Rₖ^{a}Rₖ₊₁^{a}NC(═O)—, Rₖ^{a}Rₖ₊₁^{a}NC(═S)—, Rₖ^{a}Rₖ₊₁^{a}NSO₂—, where Rₖ^{a} and Rₖ₊₁^{a} independently of each other represent “amino group substituents” the meanings of which are defined in this section, for example, hydrogen, alkyl, aryl, aralkyl, heteroaralkyl, heterocyclyl or heteroaryl, or Rₖ^{a} and Rₖ₊₁^{a} together with the N-atom, they are attached to, form through Rₖ^{a} and Rₖ₊₁^{a} 4-7-membered heterocyclyl or heterocyclenyl. The preferred alkyl groups are methyl, trifluoromethyl, cyclopropylmethyl, cyclopentylmethyl, ethyl, n-propyl, iso-propyl, n-butyl, tert.-butyl, n-pentyl, 3-pentyl, methoxyethyl, carboxymethyl, methoxycarbonylmethyl, ethoxycarbonylmethyl, benzyloxycarbonylmethyl and pyridylmethyloxycarbonylmethyl. The preferred “alkyl substituents” are cycloalkyl, aryl, heteroaryl, heterocyclyl, hydroxy, alkoxy, alkoxycarbonyl, aralkoxy, aryloxy, alkylthio, heteroarylthio, aralkylthio, alkylsulfonyl, arylsulfonyl, alkoxycarbonyl, aralkoxycarbonyl, heteroaralkyloxycarbonyl or Rₖ^{a}Rₖ₊₁^{a}N—, Rₖ^{a}Rₖ₊₁^{a}NC(═O)—, annelated arylheterocyclenyl, annelated arylheterocyclyl.

“Alkylamino” means CₙH₂ₙ₊₁NH— or (CₙH₂ₙ₊₁)(CₙH₂ₙ₊₁)N— group, in which alkyl is defined in this section. The preferred alkylamino groups are methylamino, ethylamino, n-propylamino, iso-propylamino and n-butylamino.

“Alkyloxy (alkoxy)” means CₙH₂ₙ₊₁O— group, in which alkyl is defined in this section. The preferred alkyloxy groups are methyloxy, ethyloxy, n-propyloxy, iso-propyloxy and n-butoxy.

“Alkyloxycarbonyl (alkoxycarbonyl)” means—C(O)OCₙH₂ₙ₊₁ group, in which alkyl is defined in this section.

“Amino group” means Rₖ^{a}Rₖ₊₁^{a}N— group, substituted or unsubstituted with “amino group substituent” Rₖ^{a} and Rₖ₊₁^{a}, the meanings of which are defined in this section, for example, amino (H₂N—), methylamino, diethylamino, pyrrolidino, morpholino, benzylamino or phenethylamino.

“Aminocarbonyl” means C(═O)NRₖ^{a}Rₖ₊₁^{a} group, substituted or unsubstituted with “carbamoyl substituents” Rₖ^{a} and Rₖ₊₁^{a} including hydrogen, alkenyl, alkyl, aryl, heteroaryl, heterocyclyl the meanings of which are defined in this section.

“Antagonists” mean ligands which being bound to definite receptors do not cause active cellular responses. Antagonists prevent linkage between agonists and receptors and by that block specific receptor signal transmission.

“Aryl” means an aromatic mono- or polycyclic system with 6-14 carbon atoms, predominantly 6-10 carbon atoms. Aryl may have one or more “cyclic system substituents” of the same or different structure. Phenyl, substituted phenyl, naphthyl, or substituted naphthyl are the representatives of aryl groups. Aryl could be annelated with nonaromatic cyclic system or heterocycle.

“Arylsulfonyl” means aryl-SO₂— group, in which the meaning of aryl is defined in this section.

“Bioisosteric compound” is a compound derived by means of replacement of one or more atoms in a chemical structure by other atom or group of atoms. The purpose of bioisosteric exchange is synthesis of novel compounds with enhanced biological properties without significant changes in chemical structure. Bioisosteric substitution could be physico-chemical or topologic.

“Halogen” means fluorine, chlorine, bromine and iodine. Preference is given to fluorine, chlorine and bromine.

“Heteroaryl” means an aromatic mono- or polycyclic system with 5-14 carbon atoms, preferably from 5 to 10, wherein one or more carbon atoms are substituted by one or more heteroatoms, such as N, S or O. Prefix “aza”, “oxa” or “thia” before “heteroaryl” means that N, O or S atoms are introduced in the appropriate cyclic fragment. N-Atom of heteroaryl cycle could be oxidized to N-oxide. Heteroaryl may have one or more “cyclic system substituents” of the same or different structure. Pyrrolyl, furanyl, thienyl, pyridyl, pyrazinyl, pyrimidinyl, isoxazolyl, isothiazolyl, tetrazolyl, oxazolyl, thiazolyl, pyrazolyl, furazanyl, triazolyl, 1,2,4-thiadiazolyl, pyridazinyl, quinoxalinyl, phthalazinyl, imidazo[1,2-a]pyridinyl, imidazo[2,1-b]thiazolyl, benzofurazanyl, indolyl, azaindolyl, benzoimidazolyl, benzothiazenyl, quinolinyl, imidazolyl, thienopyridyl, quinazolinyl, thienopyrimidinyl, pyrrolopyridinyl, imidazopyridinyl, isoquinolinyl, benzoazaindolyl, 1,2,4-triazinyl, thienopyrrolyl, furopyrrolyl and others are the representatives of heteroaryl radicals.

“Heterocyclenyl” means a saturated monocyclic or polycyclic system with 3-13 carbon atoms, preferably from 5 to 13 carbon atoms, in which one or more carbon atoms are substituted by heteroatom such as N, O, or S, and which comprises at least one C═C double bond or C═N double bond. Prefix “aza”, “oxa” or “thia” before “heterocyclenyl” means that N, O or S atoms are introduced in the cyclic system, respectively. Heterocyclenyl may have one or more “cyclic system substituents” of the same or different structure. N— and S— atoms of “heterocyclenyl” could be oxidized to N-oxide, S-oxide or S-dioxide. 1,2,3,4-Tetrahydropyridinyl, 1,2-dihydropyridinyl, 1,4-dihydropyridinyl, 2-pyrrolinyl, 3-pyrrolinyl, 2-imidazolyl, 2-pyrazolinyl, dihydrofuranyl, dihydrothiophenyl and others are the representatives of heterocyclenyls.

“Heterocyclyl” means an aromatic or saturated mono- or polycyclic system with 3-10 carbon atoms, preferably from 5 to 6, wherein one or more carbon atoms are substituted by one or more heteroatoms, such as N, S or O. Prefix “aza”, “oxa” or “thia” before “heterocyclyl” means that N, O or S atoms are introduced in the cycle, respectively. Heterocyclyl may have one or more “cyclic system substituents” of the same or different structure. Heterocyclyl may have one or more “cyclic system substituents” of the same or different structure. N— and S— atoms of heterocyclyl could be oxidized to N-oxide, S-oxide or S-dioxide. Piperidinyl, pyrrolidinyl, piperazinyl, morpholinyl, thiomorpholinyl, thiazolidinyl, 1,4-dioxane-2-yl, tetrahydrofuranyl, tetrahydrothiophenyl and others are examples of heterocyclyl.

“Hydrate” means stoichiometric or nonstoichiometric compositions of compounds or their salts with water.

“Bile acids” (endogenous bile acids, BA)—The main types of bile acids found in a human body are so named primary bile acids (secreted primarily by liver): cholic acid (3α, 7α, 12α-trihydroxy-5β-cholanic acid) and chenodeoxycholic (3α, 7α-dihydroxy-5β-cholanic acid), and also secondary (are formed from primary bile acids in the large gut under the influence of gut organisms): desoxycholeic acid (3α, 12α-dihydroxy-5β-cholanic acid), lithocholic (3α-monooxy-5β-cholanic acid), allocholic and ursodeoxycholic acids. From the secondary bile acids only desoxycholeic acid takes part in enterohepatic cycle at the physiologically determining level, which is then soaked in blood and secreted as a part of bile by liver. Allocholic, ursodeoxycholic and lithocholic acids are stereoisomers of cholanic and deoxycholic acids. All human bile acids have 24 carbon atoms in their structure. Bile acids are intended for stimulation of incretin hormones.

“Substituent” means a chemical radical attached to a scaffold (fragment), for example, “alkyl substituent”, “amino group substituent”, “carbamoyl substituent”, and “cyclic system substituent”, the meanings of which are defined in this section.

“Amino group substituent” means a substituent attached to amino group. Hydrogen, alkyl, cycloalkyl, aryl, heteroaryl, heterocyclyl, acyl, aroyl, alkylsulfonyl, arylsulfonyl, heteroarylsulfonyl, alkylamino carbonyl, arylamino carbonyl, heteroarylaminocarbonyl, heterocyclylaminocarbonyl, alkylaminothiocarbonyl, arylaminothiocarbonyl, heteroarylaminothiocarbonyl, heterocyclylaminothiocarbonyl, annelated heteroarylcycloalkenyl, annelated heteroarylcycloalkyl, annelated heteroarylheterocyclenyl, annelated heteroarylheterocyclenyl, annelated arylcycloalkenyl, annelated arylcycloalkyl, annelated arylheterocyclenyl, annelated arylheterocyclyl, alkoxycarbonylalkyl, aralkoxycarbonylalkyl, heteroaralkyloxycarbonylalkyl are amino group substituents.

“Carbamoyl substituent” means a substituent attached to aminocarbonyl group the meaning of which is defined in this section. Carbamoyl substituent represents hydrogen, alkyl, cycloalkyl, aryl, heteroaryl, heterocyclyl, alkoxycarbonylalkyl, aralkoxycarbonylalkyl, heteroaralkyloxycarbonylalkyl or Rₖ^{a}Rₖ₊₁^{a}N—, Rₖ^{a}Rₖ₊₁NC(═O)-alkyl, annelated heteroarylcycloalkenyl, annelated heteroarylcycloalkyl, annelated heteroarylheterocyclenyl, annelated heteroarylheterocyclyl, annelated arylcycloalkenyl, annelated arylcycloalkyl, annelated arylheterocyclenyl, annelated arylheterocyclyl. The preferred “carbamoyl substituents” are alkyl, cycloalkyl, aryl, heteroaryl, heterocyclyl, alkoxycarbonylalkyl, aralkoxycarbonylalkyl, heteroaralkyloxycarbonylalkyl or Rₖ^{a}Rₖ₊₁^{a}N—, Rₖ^{a}Rₖ₊₁NC(═O)— alkyl, annelated arylheterocyclenyl, annelated arylheterocyclyl.

“Cyclic system substituent” means a substituent attached to an aromatic or saturated cyclic system and implies hydrogen, alkyl, alkenyl, alkynyl, aryl, heteroaryl, aralkyl, heteroaralkyl, hydroxy, hydroxyalkyl, amino, aminoalkyl, alkoxy, aryloxy, acyl, aroyl, halogen, nitro, cyano, carboxy, alkoxycarbonyl, aryloxycarbonyl, aralkoxycarbonyl, alkyloxyalkyl, aryloxyalkyl, heterocyclyloxyalkyl, arylalkyloxyalkyl, heterocyclylalkyloxyalkyl, alkylsulfonyl, arylsulfonyl, heterocyclylsulfonyl, alkylsulfinyl, arylsulfinyl, heterocyclylsulfinyl, alkylthio, arylthio, heterocyclylthio, alkylsulfonylalkyl, arylsulfonylalkyl, heterocyclylsulfonylalkyl, alkylsulfinylalkyl, arylsulfinylalkyl, heterocyclylsulfinylalkyl, alkylthio alkyl, arylthioalkyl, heterocyclylthioalkyl, arylalkylsulfonylalkyl, heterocyclylalkylsulfonylalkyl, arylalkylthioalkyl, heterocyclylalkylthioalkyl, cycloalkyl, cycloalkenyl, heterocyclyl, heterocyclenyl, amidino, Rₖ^{a}Rₖ₊₁^{a}N—, Rₖ^{a}N═, Rₖ^{a}Rₖ₊₁^{a}N-alkyl-, Rₖ^{a}Rₖ₊₁^{a}NC(═O)— or Rₖ^{a}Rₖ₊₁^{a}NSO₂—, where Rₖ^{a} and Rₖ₊₁^{a} represent independently of each other “amino group substituents” the meanings of which are defined in this section, for example, hydrogen, optionally substituted alkyl, optionally substituted aryl, optionally substituted aralkyl, or optionally substituted heteroaralkyl, or a substituent Rₖ^{a}Rₖ₊₁^{a}N—, in which Rₖ^{a} may be acyl or aroyl, the meaning of Rₖ₊₁^{a} is defined above, or “cyclic system substituent” represents Rₖ^{a}Rₖ₊₁^{a}NC(═O)— or Rₖ^{a}Rₖ₊₁^{a}NSO₂—, in which Rₖ^{a} and Rₖ₊₁^{a} together with the N-atom they are attached to form through Rₖ^{a} and Rₖ₊₁^{a} 4-7 membered hererocyclyl or heterocyclenyl.

“Carboxyl” means group-CO₂H.

“Combined medicament” represents a number of drug substances for simultaneous use in the form of tablets, capsules, injections, ointments, rectal suspensions, gels and other ready forms intended for restoration, improvement or modification of physiological functions at humans and animals, and for treatment and prophylaxis of diseases, diagnostics, anesthesia, contraception, cosmetology and others. Drug substances in one unit could be presented in the form of various ready forms intended for administration into the body of animal or human by different ways, for example, peroral and rectal.

“Ligand” (from Latin ligo) represents a chemical compound (small molecule, peptide, protein, inorganic ion, and so on) capable to interact with receptors which convert this interaction into specific signal.

“Neurodegenerative diseases” mean specific conditions and diseases, accompanied by damage and primary destruction of nervous cell populations in certain areas of central nervous system. Neurodegenerative diseases include but are not limited by Alzheimer's disease, Parkinson's and Huntington's diseases (chorea); multiocular sclerosis; cerebellar degeneracy; amyotrophic lateral sclerosis; dementias with Lewy bodies; spinal muscular atrophy; peripherical neuropathy; spongy encephalitis (Creutzfeld-Jakob Disease); AIDS dementia; multi-infract dementia; frontotemporal dementias; leukoencephalopathy (spongy degeneration of white matter); chronic neurodegenerative diseases; insult; ischemic, reperfusion and hypoxic brain damage; epilepsy; cerebral ischemia; glaucoma; traumatic brain injury; Down's syndrome; encephalomyelitis; meningitis; encephalitis; neuroblastoma; schizophrenia; depression. Moreover, neurodegenerative diseases include pathological states and disorders associated with hypoxia, substance abuse, causing dependability, under neurotoxins influence; infectious and oncological brain diseases as well as neuronal damages associated with autoimmune and endocrine diseases and others.

“Optionally substituted radical” means a radical either without substituents or containing one or more substituents.

“Lower alkyl” means straight or branched alkyl with 1-4 carbon atoms.

“Receptors” (from latin recipere) represent biological macromolecules located either on cytoplasmic cell membrane or intracellular, capable specifically interact with restricted number of physiologically active compounds (ligands) and transform signal of this interaction into definite cellular response.

“TGR5 receptors” (known also as GPBAR1, M-BAR, AXOR 109 and GPCR19) are GPCR-receptors, bound to Gs-proteins. They are mainly expressed in gastrointestinal tract, gall bladder, spleen, lungs and placenta.

“Cycloalkyl” means saturated mono- or polycyclic system with 3-10 carbon atoms. Cycloalkyl may have one or more “cyclic system substituents” of the same or different structure. Cyclopropyl, cyclobutyl, cyclopentyl, cyclohexyl, decalinyl, norbornyl, adamant-1-yl and others are the representatives of cycloalkyl groups. Cycloalkyl could be annelated with aromatic cycle or heterocycle. Alkyl, aralkoxy, hydroxy or Rₖ^{a}Rₖ₊₁^{a}N— are preferred “cyclic system substituents”, the meanings of which are defined in this section.

“Pharmaceutical composition” means a composition comprising compound of the general formula 1 and at least one of components selected from the group consisting of pharmaceutically acceptable and pharmacologically compatible fillers, solvents, diluents, auxiliary, distributing and sensing agents, delivery agents, such as preservatives, stabilizers, disintegrators, moisteners, emulsifiers, suspending agents, thickeners, sweeteners, flavouring agents, aromatizing agents, antibacterial agents, fungicides, lubricants, and prolonged delivery controllers, choice and suitable proportions of which depend on nature and way of administration and dosage. Examples of suitable suspending agents are ethoxylated isostearyl alcohol, polyoxyethene, sorbitol and sorbitol ether, microcrystalline cellulose, aluminum metahydroxide, bentonite, agar-agar and tragacant and their mixtures as well. Protection against action of microorganisms can be provided by various antibacterial and antifungal agents, such as, for example, parabens, chlorobutanole, sorbic acid, and similar compounds. Composition may also contain isotonic agents, such as, for example, sugar, sodium chloride, and similar compounds. Prolonged effect of composition may be achieved by agents slowing down absorption of active ingredient, for example, aluminum monostearate and gelatine. Examples of suitable carriers, solvents, diluents and delivery agents include water, ethanol, polyalcohols and their mixtures, natural oils (such as olive oil) and organic esters (such as ethyl oleate) for injections. Examples of fillers are lactose, milk-sugar, sodium citrate, calcium carbonate, calcium phosphate and the like. Examples of disintegrators and distributors are starch, alginic acid and its salts, and silicates. Examples of suitable lubricants are magnesium stearate, sodium lauryl sulfate, talc and polyethylene glycol of high molecular weight. Pharmaceutical composition for peroral, sublingval, transdermal, intramuscular, intravenous, subcutaneous, local or rectal administration of active ingredient, alone or in combination with another active compound may be administered to humans and animals in standard administration form, or in mixture with traditional pharmaceutical carriers. Suitable standard administration forms include peroral forms such as tablets, gelatin capsules, pills, powders, granules, chewing-gums and peroral solutions or suspensions, for example, therapeutic kit; sublingval and transbuccal administration forms; aerosols; implants; local, transdermal, subcutaneous, intramuscular, intravenous, intranasal or intraocular forms and rectal administration forms.

As a rule, pharmaceutical compositions are prepared by means of standard procedure involving mixing of active compounds with liquid or overgrounded solid carrier. For suppository preparation besides active components cacao oil, its alloys with paraffin and hydrogenated fats, vegetable and animal hydrogenated fats, solid fat, lanol, hydrogenated fat alloys with wax, solid paraffin and other bases allowed for medical use are also employed.

“Pharmaceutically acceptable excipients” mean diluents, adjuvant agents and/or carriers employing in the sphere of pharmaceutics.

“Pharmaceutically acceptable salt” means relatively nontoxic both organic and inorganic salts of acids and bases disclosed in this invention. Salts could be prepared in situ in the processes of synthesis, isolation or purification of compounds or they could be prepared specially. In particular, salts of bases could be prepared from purified base of the disclosed compound and suitable organic or mineral acid. Examples of salts prepared in this manner include hydrochlorides, hydrobromides, sulfates, bisulfates, phosphates, nitrates, acetates, oxalates, valeriates, oleates, palmitates, stearates, laurates, borates, benzoates, lactates, p-toluenesulfonates, citrates, maleates, fumarates, succinates, tartrates, methane sulphonates, malonates, salicylates, propionates, ethane sulphonates, benzene sulfonates, sulfamates and the like (Detailed description of such salts properties is given in: Berge S. M., et al., “Pharmaceutical Salts” J. Pharm. Sci., 1977, 66: 1-19). Salts of the disclosed acids may also be prepared by the reaction of purified acids specifically with suitable base; moreover, metal salts and amine salts may be synthesized too. Metal salts are salts of sodium, potassium, calcium, barium, magnesium, lithium and aluminum, sodium and potassium salts being preferred. Suitable inorganic bases from which metal salts can be prepared are sodium hydroxide, carbonate, bicarbonate and hydride; potassium hydroxide, carbonate and bicarbonate, lithium hydroxide, calcium hydroxide, magnesium hydroxide, zinc hydroxide. Organic bases suitable for preparation of the disclosed acid salts are amines and amino acids of sufficient basicity to produce stable salt suitable for medical purposes use (in particular, they are to have low toxicity). Such amines include ammonia, methylamine, dimethylamine, trimethylamine, ethylamine, diethylamine, triethylamine, benzylamine, dibenzylamine, dicyclohexylamine, piperazine, ethylpiperidine, tris(hydroxymethyl)aminomethane and the like. Besides, salts can be prepared using some tetraalkylammonium hydroxides, such as holine, tetramethylammonium, tetraethylammonium, and the like. Aminoacids may be selected from the main aminoacids—lysine, ornithine and agrinine.

The purpose of the present invention is novel derivatives of 2,3,4,5-tetrahydrobenzo[f][1,4]oxazepines which are TGR5 receptor agonists.

The purpose in view is achieved by derivatives of compounds of the general formula 1, or pharmaceutically acceptable salts thereof,

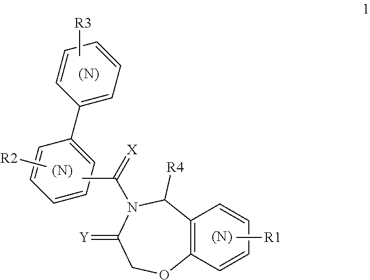

wherein:

R1, R2 and R3 independently of each other are hydrogen, a C₁-C₃ alkyl, halogen, a trifluoromethyl group, a C₁-C₃ alkoxy, a cyano group, a trifluoromethoxy group; an amino group substituted with C₁-C₄ alkyl; or two radicals R3 at the adjacent carbon atoms of the benzene ring, together with this benzene ring form 3,4-methylenedioxyphenyl;

R4 is hydrogen, a C₁-C₅ alkyl, a carboxyl group, a C₁-C₃ alkoxycarbonyl or an amide group CONHR5;

R5 is an optionally substituted C₁-C₃ alkyl, a C₅-C₆ cycloalkyl, an optionally substituted phenyl, benzyl, pyridyl;

X and Y are two hydrogen atoms or an oxygen, provided that

Y═O and X=2H, or Y=2H and X═O, or X═Y=2H;

(N)—denotes that benzene ring could be bioisosterically replaced by azaheterocyclic ring such as: pyridine, pyrimidine, pyridazine, triazine or pyrazine.

The preferable compounds are derivatives of compounds of the general formulas 1.1 and 1.2, or pharmaceutically acceptable salts thereof,

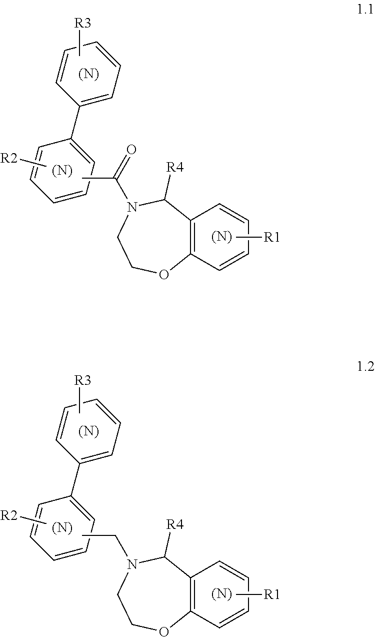

wherein:

R1, R2, R3, R4 and (N) have the above meanings.

The more preferred compounds are derivatives of compounds of the general formulas 1.3 and 1.4 or pharmaceutically acceptable salts thereof,

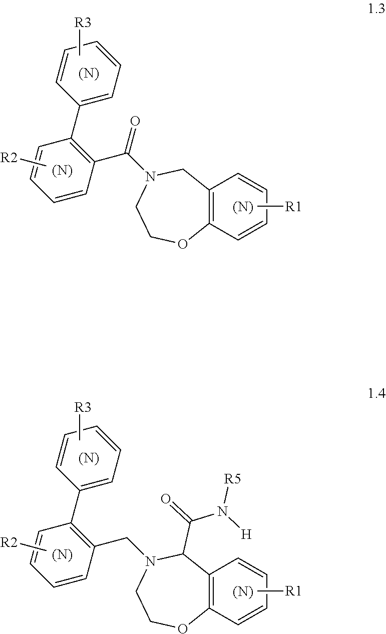

wherein:

R1, R2, R3, R5 and (N) have the above meanings.

The most preferable compounds are compounds represented by formulas 01-101:
4-(3-phenylbenzyl)-7-trifluoromethyl-2,3,4,5-tetrahydrobenzo[f][1,4]oxazepine (01),
4-(3-phenylbenzoyl)-7-trifluoromethyl-2,3,4,5-tetrahydrobenzo[f][1,4]oxazepine (02),
4-(4-phenylbenzoyl)-7-trifluoromethyl-2,3,4,5-tetrahydrobenzo[f][1,4]oxazepine (03),
4-(2-phenylbenzoyl)-7-trifluoromethyl-2,3,4,5-tetrahydrobenzo[f][1,4]oxazepine (04),
4-[2-(pyridin-4-yl)benzoyl]-7-fluoro-2,3,4,5-tetrahydrobenzo[f][1,4]oxazepine (05),
4-[2-(pyridin-2-yl)benzoyl]-7-fluoro-2,3,4,5-tetrahydrobenzo[f][1,4]oxazepine (06),
4-(4-phenylnicotinoyl)-7-trifluoromethyl-2,3,4,5-tetrahydrobenzo[f][1,4]oxazepine (07),
4-[(4-phenylpyridin-3-yl)methyl]-7-trifluoromethyl-2,3,4,5-tetrahydrobenzo[f][1,4]oxazepine (08),
4-[2-(pyridin-4-yl)benzyl]-7-fluoro-2,3,4,5-tetrahydrobenzo[f][1,4]oxazepine (09),
4-[2-(pyridin-2-yl)benzyl]-7-fluoro-2,3,4,5-tetrahydrobenzo[f][1,4]oxazepine (10),
4-(4-phenylnicotinoyl)-7-methyl-2,3,4,5-tetrahydrobenzo[f][1,4]oxazepine (11),
4-[2-(pyridin-4-yl)benzoyl]-7-methyl-2,3,4,5-tetrahydrobenzo[f][1,4]oxazepine (12),
4-[2-(pyridin-2-yl)benzoyl]-7-methyl-2,3,4,5-tetrahydrobenzo[f][1,4]oxazepine (13),
4-(3-pheylbenzyl)-7-trifluoromethyl-2,3,4,5-tetrahydrobenzo[f][1,4]oxazepine-5-carboxylic acid cyclopentylamide (14),
4-(3-phenylbenzyl)-7-trifluoromethyl-2,3,4,5-tetrahydrobenzo[f][1,4]oxazepine-5-carboxylic acid cyclohexylamide (15),
4-(4-phenylbenzyl)-7-trifluoromethyl-2,3,4,5-tetrahydrobenzo[f][1,4]oxazepine-5-carboxylic acid (16),
4-(2-phenylbenzyl)-7-trifluoromethyl-2,3,4,5-tetrahydrobenzo[f][1,4]oxazepine-5-carboxylic acid tert-butylamide (17),
4-[2-(pyridin-4-yl)benzyl]-7-fluoro-2,3,4,5-tetrahydrobenzo[f][1,4]oxazepine-5-carboxylic acid tert-butylamide (18),
4-[2-(pyridin-2-yl)benzyl]-7-fluoro-2,3,4,5-tetrahydrobenzo[f][1,4]oxazepine-5-carboxylic acid tert-butylamide (19),
4-(2-phenylbenzyl)-7-methoxy-2,3,4,5-tetrahydrobenzo[f][1,4]oxazepine-5-carboxylic acid tert-butylamide (20),
4-[2-(pyridin-4-yl)benzyl]-7-methoxy-2,3,4,5-tetrahydrobenzo[f][1,4]oxazepine-5-carboxylic acid tert-butylamide (21),
4-[2-(pyridin-2-yl)benzyl]-7-methoxy-2,3,4,5-tetrahydrobenzo[f][1,4]oxazepine-5-carboxylic acid tert-butylamide (22),
4-[2-(3,4-methylenedioxyphenyl)benzyl]-2,3,4,5-tetrahydrobenzo[f][1,4]oxazepine-5-carboxylic acid tert-butylamide (23),
4-[2-(pyridin-4-yl)benzyl]-2,3,4,5-tetrahydrobenzo[f][1,4]oxazepine-5-carboxylic acid tert-butylamide (24),
4-[2-(pyridin-2-yl)benzyl]-2,3,4,5-tetrahydrobenzo[f][1,4]oxazepine-5-carboxylic acid tert-butylamide (25),
4-[2-(pyridin-3-yl)benzyl]-2,3,4,5-tetrahydrobenzo[f][1,4]oxazepine-5-carboxylic acid tert-butylamide (26),
4-[2-(3,4-methylenedioxyphenyl)benzyl]-2,3,4,5-tetrahydrobenzo[f][1,4]oxazepine-5-carboxylic acid (27),
4-[2-(pyridin-4-yl)benzyl]-2,3,4,5-tetrahydrobenzo[f][1,4]oxazepine-5-carboxylic acid (28),
4-[2-(pyridin-2-yl)benzyl]-2,3,4,5-tetrahydrobenzo[f][1,4]oxazepine-5-carboxylic acid (29),
4-[2-(pyridin-3-yl)benzyl]-2,3,4,5-tetrahydrobenzo[f][1,4]oxazepine-5-carboxylic acid (30),
4-[2-(3,4-methylenedioxyphenyl)benzyl]-2,3,4,5-tetrahydrobenzo[f][1,4]oxazepine-5-carboxylic acid ethyl ester (31),
4-[2-(pyridin-4-yl)benzyl]-2,3,4,5-tetrahydrobenzo[f][1,4]oxazepine-5-carboxylic acid ethyl ester (32),
4-[2-(pyridin-2-yl)benzyl]-2,3,4,5-tetrahydrobenzo[f][1,4]oxazepine-5-carboxylic acid ethyl ester (33),
4-[2-(pyridin-3-yl)benzyl]-2,3,4,5-tetrahydrobenzo[f][1,4]oxazepine-5-carboxylic acid ethyl ester (34),
4-[2-(4-methoxyphenyl)benzyl]-2,3,4,5-tetrahydrobenzo[f][1,4]oxazepine-5-carboxylic acid tert-butylamide (35),
4-[2-(3-methoxyphenyl)benzyl]-2,3,4,5-tetrahydrobenzo[f][1,4]oxazepine-5-carboxylic acid tert-butylamide (36),
4-[2-(2-methoxyphenyl)benzyl]-2,3,4,5-tetrahydrobenzo[f][1,4]oxazepine-5-carboxylic acid tert-butylamide (37),
4-[2-(4-methylphenyl)benzyl]-2,3,4,5-tetrahydrobenzo[f][1,4]oxazepine-5-carboxylic acid tert-butylamide (38),
4-[2-(3-methylphenyl)benzyl]-2,3,4,5-tetrahydrobenzo[f][1,4]oxazepine-5-carboxylic acid tert-butylamide (39),
4-[2-(2-methylphenyl)benzyl]-2,3,4,5-tetrahydrobenzo[f][1,4]oxazepine-5-carboxylic acid tert-butylamide (40),
4-[2-(4-fluorophenyl)benzyl]-2,3,4,5-tetrahydrobenzo[f][1,4]oxazepine-5-carboxylic acid tert-butylamide (41),
4-[2-(3-fluorophenyl)benzyl]-2,3,4,5-tetrahydrobenzo[f][1,4]oxazepine-5-carboxylic acid tert-butylamide (42),
4-[2-(2-fluorophenyl)benzyl]-2,3,4,5-tetrahydrobenzo[f][1,4]oxazepine-5-carboxylic acid tert-butylamide (43),
4-[2-(4-chlorophenyl)benzyl]-2,3,4,5-tetrahydrobenzo[f][1,4]oxazepine-5-carboxylic acid tert-butylamide (44),
4-[2-(3-chlorophenyl)benzyl]-2,3,4,5-tetrahydrobenzo[f][1,4]oxazepine-5-carboxylic acid tert-butylamide (45),
4-[2-(2-chlorophenyl)benzyl]-2,3,4,5-tetrahydrobenzo[f][1,4]oxazepine-5-carboxylic acid tert-butylamide (46),
4-[2-(4-methylphenyl)benzyl]-7-fluoro-2,3,4,5-tetrahydrobenzo[f][1,4]oxazepine-5-carboxylic acid tert-butylamide (47),
4-[2-(3-methylphenyl)benzyl]-7-fluoro-2,3,4,5-tetrahydrobenzo[f][1,4]oxazepine-5-carboxylic acid tert-butylamide (48),
4-[2-(2-methylphenyl)benzyl]-7-fluoro-2,3,4,5-tetrahydrobenzo[f][1,4]oxazepine-5-carboxylic acid tert-butylamide (49),
4-[2-(4-fluorophenyl)benzyl]-7-fluoro-2,3,4,5-tetrahydrobenzo[f][1,4]oxazepine-5-carboxylic acid tert-butylamide (50),
4-[2-(3-fluorophenyl)benzyl]-7-fluoro-2,3,4,5-tetrahydrobenzo[f][1,4]oxazepine-5-carboxylic acid tert-butylamide (51),
4-[2-(2-fluorophenyl)benzyl]-7-fluoro-2,3,4,5-tetrahydrobenzo[f][1,4]oxazepine-5-carboxylic acid tert-butylamide (52),
4-[2-(pyrimidin-4-yl)benzyl]-2,3,4,5-tetrahydrobenzo[f][1,4]oxazepine-5-carboxylic acid tert-butylamide (53),
4-[2-(pyrimidin-2-yl)benzyl]-2,3,4,5-tetrahydrobenzo[f][1,4]oxazepine-5-carboxylic acid tert-butylamide (54),
4-[2-(pyrimidin-5-yl)benzyl]-2,3,4,5-tetrahydrobenzo[f][1,4]oxazepine-5-carboxylic acid tert-butylamide (55),
4-[2-(pyrazin-2-yl)benzyl]-2,3,4,5-tetrahydrobenzo[f][1,4]oxazepine-5-carboxylic acid tert-butylamide (56),
4-[2-(pyrazin-2-yl)benzyl]-2,3,4,5-tetrahydrobenzo[f][1,4]oxazepine-5-carboxylic acid cyclopentylamide (57),
4-[2-(pyrazin-2-yl)benzyl]-7-fluoro-2,3,4,5-tetrahydrobenzo[f][1,4]oxazepine-5-carboxylic acid cyclopentylamide (58),
4-[2-(pyrazin-2-yl)benzyl]-7-fluoro-2,3,4,5-tetrahydrobenzo[f][1,4]oxazepine-5-carboxylic acid (59),
4-[2-(pyrimidin-4-yl)benzyl]-2,3,4,5-tetrahydrobenzo[f][1,4]oxazepine-5-carboxylic acid (60),
4-[2-(pyrimidin-2-yl)benzyl]-2,3,4,5-tetrahydrobenzo[f][1,4]oxazepine-5-carboxylic acid (61),
4-[2-(pyrimidin-5-yl)benzyl]-2,3,4,5-tetrahydrobenzo[f][1,4]oxazepine-5-carboxylic acid (62),
4-[2-(4-methoxyphenyl)benzyl]-2,3,4,5-tetrahydrobenzo[f][1,4]oxazepine-5-carboxylic acid (63),
4-[2-(3-methoxyphenyl)benzyl]-2,3,4,5-tetrahydrobenzo[f][1,4]oxazepine-5-carboxylic acid (64),
4-[2-(2-methoxyphenyl)benzyl]-2,3,4,5-tetrahydrobenzo[f][1,4]oxazepine-5-carboxylic acid (65),
4-[2-(4-methylphenyl)benzyl]-2,3,4,5-tetrahydrobenzo[f][1,4]oxazepine-5-carboxylic acid (66),
4-[2-(3-methylphenyl)benzyl]-2,3,4,5-tetrahydrobenzo[f][1,4]oxazepine-5-carboxylic acid (67),
4-[2-(2-methylphenyl)benzyl]-2,3,4,5-tetrahydrobenzo[f][1,4]oxazepine-5-carboxylic acid (68),
4-[2-(4-fluorophenyl)benzyl]-2,3,4,5-tetrahydrobenzo[f][1,4]oxazepine-5-carboxylic acid (69),
4-[2-(3-fluorophenyl)benzyl]-2,3,4,5-tetrahydrobenzo[f][1,4]oxazepine-5-carboxylic acid (70),
4-[2-(2-fluorophenyl)benzyl]-2,3,4,5-tetrahydrobenzo[f][1,4]oxazepine-5-carboxylic acid (71),
4-[2-(4-chlorophenyl)benzyl]-2,3,4,5-tetrahydrobenzo[f][1,4]oxazepine-5-carboxylic acid (72),
4-[2-(3-chlorophenyl)benzyl]-2,3,4,5-tetrahydrobenzo[f][1,4]oxazepine-5-carboxylic acid (73),
4-[2-(2-chlorophenyl)benzyl]-2,3,4,5-tetrahydrobenzo[f][1,4]oxazepine-5-carboxylic acid (74),
4-[2-(4-fluorophenyl)benzyl]-7-fluoro-2,3,4,5-tetrahydrobenzo[f][1,4]oxazepine-5-carboxylic acid (75),
4-[2-(3-fluorophenyl)benzyl]-7-fluoro-2,3,4,5-tetrahydrobenzo[f][1,4]oxazepine-5-carboxylic acid (76),
4-[2-(2-fluorophenyl)benzyl]-7-fluoro-2,3,4,5-tetrahydrobenzo[f][1,4]oxazepine-5-carboxylic acid (77),
4-(2-phenylbenzyl)-7-trifluoromethyl-2,3,4,5-tetrahydrobenzo[f][1,4]oxazepine-5-carboxylic acid (78),
4-(2-phenylbenzyl)-7-trifluoromethoxy-2,3,4,5-tetrahydrobenzo[f][1,4]oxazepine-5-carboxylic acid (79),
4-(2-phenylbenzyl)-7-tert-butyl-2,3,4,5-tetrahydrobenzo[f][1,4]oxazepine-5-carboxylic acid (80),
4-(3-phenylbenzyl)-3-oxo-7-trifluoromethyl-2,3,4,5-tetrahydrobenzo[f][1,4]oxazepine-5-carboxylic acid cyclopentylamide (81),
4-(3-phenylbenzyl)-3-oxo-7-trifluoromethyl-2,3,4,5-tetrahydrobenzo[f][1,4]oxazepine-5-carboxylic acid cyclohexylamide (82),
4-(2-phenylbenzyl)-3-oxo-7-trifluoromethyl-2,3,4,5-tetrahydrobenzo[f][1,4]oxazepine-5-carboxylic acid tert-butylamide (83),
4-[2-(pyridin-4-yl)benzyl]-3-oxo-7-fluoro-2,3,4,5-tetrahydrobenzo[f][1,4]oxazepine-5-carboxylic acid tert-butylamide (84),
4-[2-(pyridin-2-yl)benzyl]-3-oxo-7-fluoro-2,3,4,5-tetrahydrobenzo[f][1,4]oxazepine-5-carboxylic acid tert-butylamide (85),
4-(2-phenylbenzyl)-3-oxo-7-methoxy-2,3,4,5-tetrahydrobenzo[f][1,4]oxazepine-5-carboxylic acid tert-butylamide (86),
4-[2-(pyridin-4-yl)benzyl]-3-oxo-7-methoxy-2,3,4,5-tetrahydrobenzo[f][1,4]oxazepine-5-carboxylic acid tert-butylamide (87),
4-[2-(pyridin-2-yl)benzyl]-3-oxo-7-methoxy-2,3,4,5-tetrahydrobenzo[f][1,4]oxazepine-5-carboxylic acid tert-butylamide (88),
4-[2-(3,4-methylenedioxyphenyl)benzyl]-3-oxo-2,3,4,5-tetrahydrobenzo[f][1,4]oxazepine-5-carboxylic acid tert-butylamide (89),
4-[2-(pyridin-4-yl)benzyl]-3-oxo-2,3,4,5-tetrahydrobenzo[f][1,4]oxazepine-5-carboxylic acid tert-butylamide (90),
4-[2-(pyridin-2-yl)benzyl]-3-oxo-2,3,4,5-tetrahydrobenzo[f][1,4]oxazepine-5-carboxylic acid tert-butylamide (91),
4-[2-(pyridin-3-yl)benzyl]-3-oxo-2,3,4,5-tetrahydrobenzo[f][1,4]oxazepine-5-carboxylic acid tert-butylamide (92),
4-[2-(4-methoxyphenyl)benzyl]-3-oxo-2,3,4,5-tetrahydrobenzo[f][1,4]oxazepine-5-carboxylic acid tert-butylamide (93),
4-[2-(3-methoxyphenyl)benzyl]-3-oxo-2,3,4,5-tetrahydrobenzo[f][1,4]oxazepine-5-carboxylic acid tert-butylamide (94),
4-[2-(2-methoxyphenyl)benzyl]-3-oxo-2,3,4,5-tetrahydrobenzo[f][1,4]oxazepine-5-carboxylic acid tert-butylamide (95),
4-[2-(4-methylphenyl)benzyl]-3-oxo-2,3,4,5-tetrahydrobenzo[f][1,4]oxazepine-5-carboxylic acid tert-butylamide (96),
4-[2-(3-methylphenyl)benzyl]-3-oxo-2,3,4,5-tetrahydrobenzo[f][1,4]oxazepine-5-carboxylic acid tert-butylamide (97),
4-[2-(2-methylphenyl)benzyl]-3-oxo-2,3,4,5-tetrahydrobenzo[f][1,4]oxazepine-5-carboxylic acid tert-butylamide (98),
4-[2-(4-fluorophenyl)benzyl]-3-oxo-2,3,4,5-tetrahydrobenzo[f][1,4]oxazepine-5-carboxylic acid tert-butylamide (99),
4-[2-(3-fluorophenyl)benzyl]-3-oxo-2,3,4,5-tetrahydrobenzo[f][1,4]oxazepine-5-carboxylic acid tert-butylamide (100),
4-[2-(2-fluorophenyl)benzyl]-3-oxo-2,3,4,5-tetrahydrobenzo[f][1,4]oxazepine-5-carboxylic acid tert-butylamide (101).

The subject of the present invention is a method for preparation of 2,3,4,5-tetrahydrobenzo[f][1,4]oxazepine derivatives of the general formula 1.3, consisting in reduction of the corresponding o-cyanooxyacetate of the general formula A1 over Ni/Raney with subsequent cyclization and LiAlH₄ reduction of the cyclization product that gave a compound of the general formula A2, and acylation of the latter with the suitable biphenyl acyl halogenide,

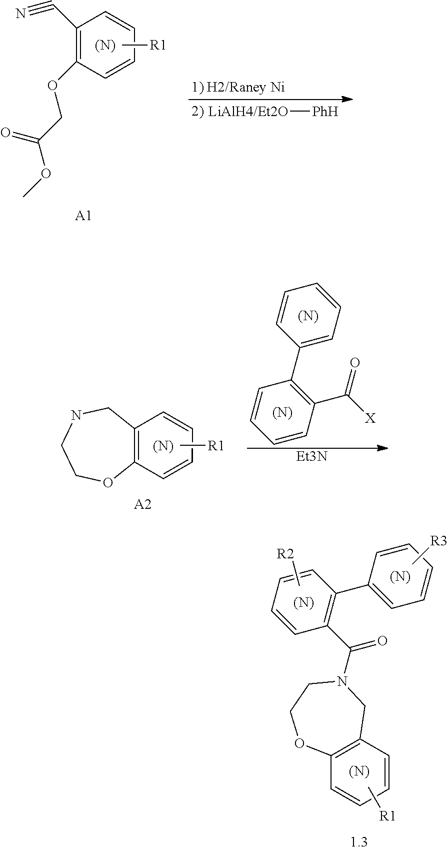

wherein:
R1, R2, R3, X and (N) have the above meanings.

The subject of the present invention is a method for preparation of the derivatives of compound of the general formula 1.4, consisting in interaction of the corresponding aldehydro-acids of the general formula B1 with isonitriles and suitable amines and reduction of the obtained compound of the general formula B2 with borane-methyl sulfide complex in tetrahydrofuran,

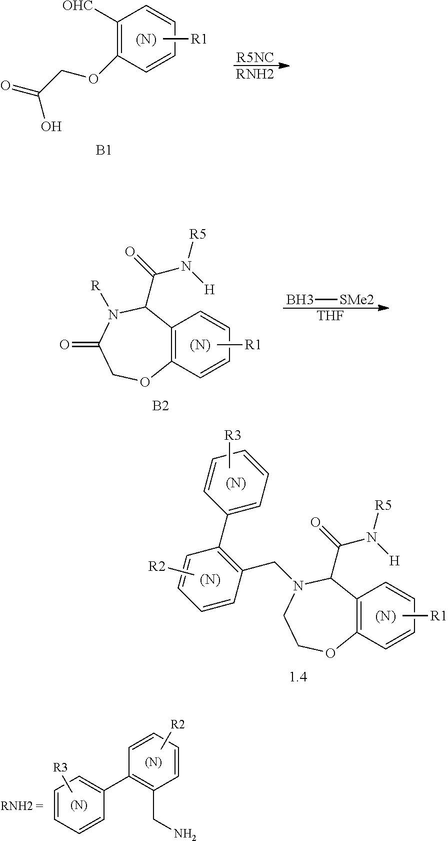

wherein:
R1, R2, R3, R5 and (N) have the above meanings.

The subject of the present invention is an active component exhibiting the property to stimulate secretion of incretin hormones, (agonist of bile acid receptorTGR5), representing derivatives of compounds of the general formula 1.

Nonsteroid agonists of BA receptors are specifically bound to BA receptors and stimulate secretion of incretin hormones (GLP1, YYP, GIP). The most preferable are bile acids TGR5 receptor agonists.

The subject of the present invention is a pharmaceutical composition comprising effective amount of active component for prophylaxis and treatment of metabolic diseases and associated with them cardiovascular and neurodegenerative diseases.

The subject of the present invention is a pharmaceutical composition for prophylaxis and treatment of metabolic diseases and associated with them cardiovascular and neurodegenerative diseases, comprising effective amount of active component and proteinase DPP-IV inhibitor, and also any of the mentioned above pharmaceutical compositions in the form of tablets, capsules or injections placed in pharmaceutically acceptable packing.

Protease DPP-IV inhibitors—are perspective agents, preventing GLP1 protein destruction by pathologic process. Their use, however, does not lead to the growth of the amount of GLP1 in organism, but to its maintenance only. The most preferable DPP-IV proteinase inhibitors are known drugs, such as: Vildagliptin, Saxagliptin, Sitagliptin, Teneligliptin, Linagliptin, Dutogliptin, Alogliptin, Gemigliptin, Carmegliptin and the like. The combined use of two heterotargeted agents joint application of which results in synergetic effect, facilitate the induction of required hormones and preservation of them.

Pharmaceutical composition may include pharmaceutically acceptable excipients. Pharmaceutically acceptable excipients mean diluents, auxiliary agents and/or carriers applied in the sphere of pharmaceutics. According to the invention pharmaceutical composition together with an active component of the general formula 1 may include other active ingredients provided that they do not give rise to undesirable effects, such as allergic reactions.

If needed, according to the present invention pharmaceutical compositions could be used in clinical practice in various forms prepared by mixing the said compositions with traditional pharmaceutical carries, for example, peroral forms (such as, tablets, gelatinous capsules, pills, solutions or suspensions); forms for injections (such as, solutions or suspensions for injections, or a dry powder for injections which only requires addition of water for injections before utilization), local forms (such as ointments or solutions).

According to the present invention the carriers used in pharmaceutical compositions represent carriers which are used in the sphere of pharmaceutics for preparation of commonly used forms. Binding agents, greasing agents, disintegrators, solvents, diluents, stabilizers, suspending agents, colorless agents, taste flavors are used for peroral forms; antiseptic agents, solubilizers, stabilizers are used in forms for injections; base materials, diluents, greasing agents, antiseptic agents are used in local forms.

A novel pharmaceutical composition could be prepared by mixing an active component representing at least one compound of the general formulas 1, 1.1, 1.2, 1.3, 1.4 or its pharmaceutically acceptable salt and/or hydrate with inert filler and/or solvent.

The subject of the present invention is a combined medicament for prophylaxis and treatment of metabolic diseases and associated with them cardiovascular and neurodegenerative diseases comprising in effective amount of proteinase DPP-IV inhibitor and a stimulator of incretin hormones secretion (agonists of bile acids TGR5 receptors)—endogenous bile acid or a mixture of bile acids, the latter in the form of rectal suspension or gel.

Distinctive feature of this combined medicament is employment of natural agonists of bile acids TGR5 receptors—endogenous bile acids or a mixture of endogenous bile acids for the purpose of stimulation of incretin hormones secretion. It is based on the property of bile acids to induce incretin synthesis. The effect is achieved by means of activation of BA receptor (TGR5, mainly) existing in L-cells membranes. Bile acids include the following BA: cholic, deoxycholic, chenodeoxycholic, glycocholic, glycodeoxycholic, glycochenodeoxycho lic, taurocholic, taurodeoxycholic, taurochenodeoxycholic, and also their conjugates with other endogenous compounds (amino acids, taurin) or their prodrugs or transport systems. Employment of taurocholic and glycodeoxycholic BA, and also their prodrugs or transport systems is the most preferable.

The subject of the present invention is a method for prophylaxis and treatment of metabolic diseases and associated with them cardiovascular and neurodegenerative diseases consisting in administration of effective amount of novel active component or novel pharmaceutical composition.

The subject of the present invention is a method for prophylaxis and treatment of metabolic diseases and associated with them cardiovascular and neurodegenerative diseases consisting in administration of effective amount of combined medicament, provided that proteinase DPP-IV inhibitor is administered peroral or by injection, and endogenous bile acid or mixture of endogenous bile acids is administered per rectum.

Because of peculiarities of metabolic landscape, endogenous BA at peroral administration do not reach their receptors in sufficient amount and do not produce required useful effect. The reason for it is the fact, that the concentration of regulatory peptides in mucus membrane of gastrointestinal tract is substantially higher in its caudal part.

Combination of several heterotargeted agents (TGR5 agonists, DPP-IV inhibitors) is suitable for treatment of the whole number of diseases, such as: diabetes, diabesity, obesity, loss of insulin sensitivity, metabolic syndrome, disorder of glucose metabolism, coronary syndrome, ventricular dysfunction, post-infarct state, nervous system diseases, neurodegenerative diseases (for example, Alzheimer's disease), insult, renal irritation, hypervolemia, ischemia, bowel diseases, osteopathy and other diseases of locomotor system.

Medicaments could be administered peroral or parenterally (for example, intravenous, subcutaneous, intraperitoneally or local). Clinical dose of active component, pharmaceutical composition or combined medicament, comprising pharmaceutically effective amount of active component, at patients may be corrected depending on: therapeutic efficiency and bio-accessibility of active ingredients in patients' organism, rate of their exchange and removal from organism, and age, gender, and severity of patient's symptoms. Thus, the daily intake for adults normally is 10˜500 mg, preferably 50˜300 mg. While preparing pharmaceutical composition as a dose unit the above effective dose is to be taken into consideration, at this each dose unit of composition contains 10˜500 mg, preferably −50˜300 mg. Following the instructions of physician or pharmacist, the medicaments may be taken several times over specified periods of time (preferably, from one to six times).

The invention is illustrated by the following drawings.

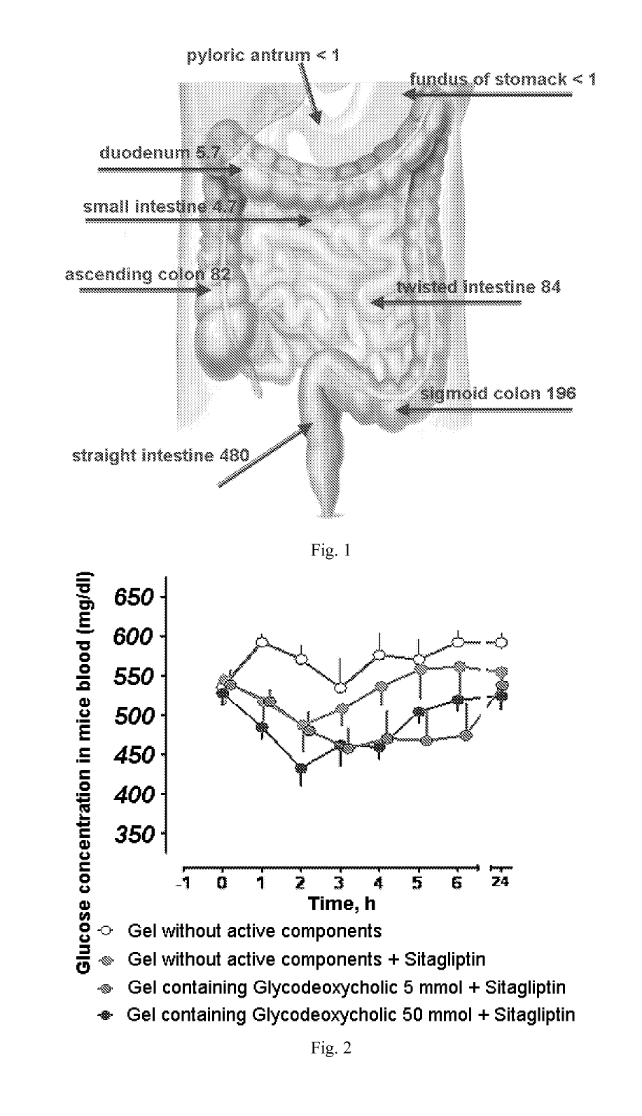

FIG. 1 Distribution of secretable PYY peptide in various parts of gastrointestinal tract showing substantial growth of its concentration in anterograde direction.

FIG. 2 Influence of rectal administration of BA GDC on blood glucose level at diabesity mice of db/db line.

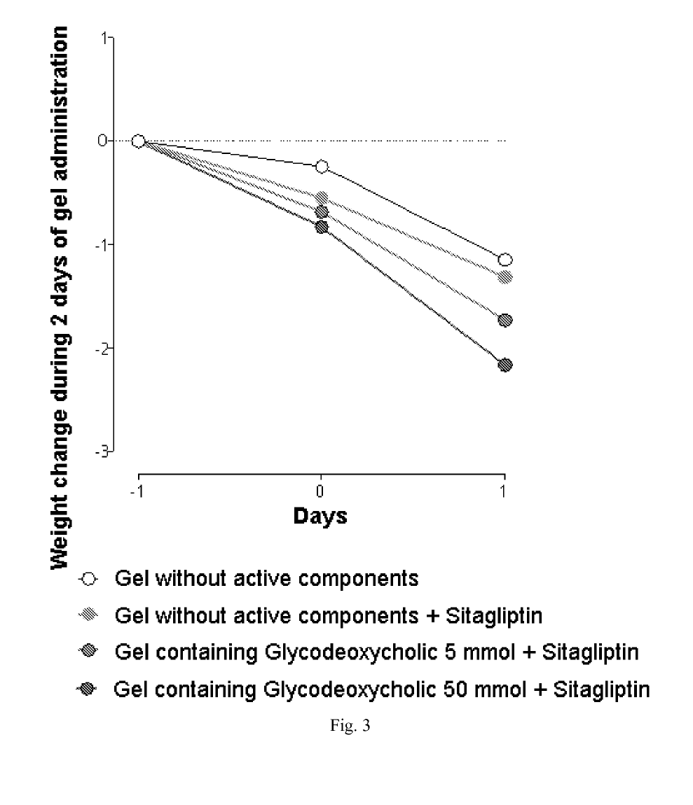

FIG. 3 Influence of rectal administration of BA GDC on weight of diabesity mice of db/db line (P<0.05).

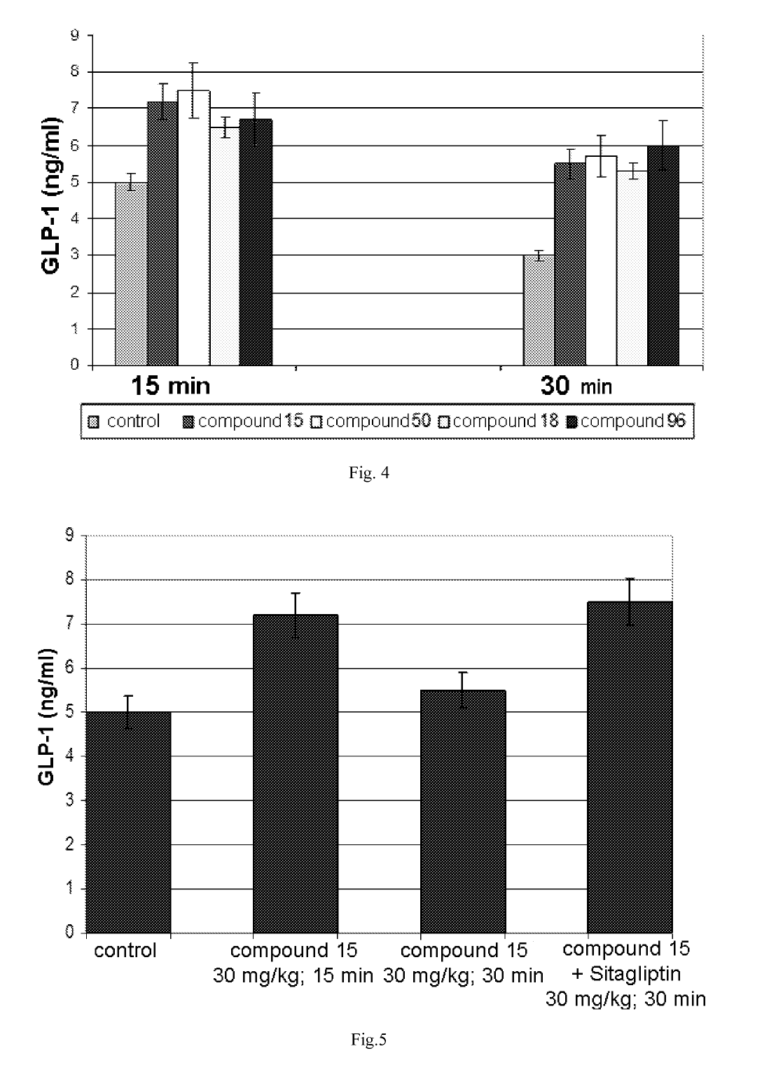

FIG. 4 Stimulation of GLP-1 secretion in 15 and 30 minutes after glucose administration.

FIG. 5 Stability of GLP-1 peptide in plasma, secretion of which was induced by the action of TGR5 receptors agonist in glucose presence.

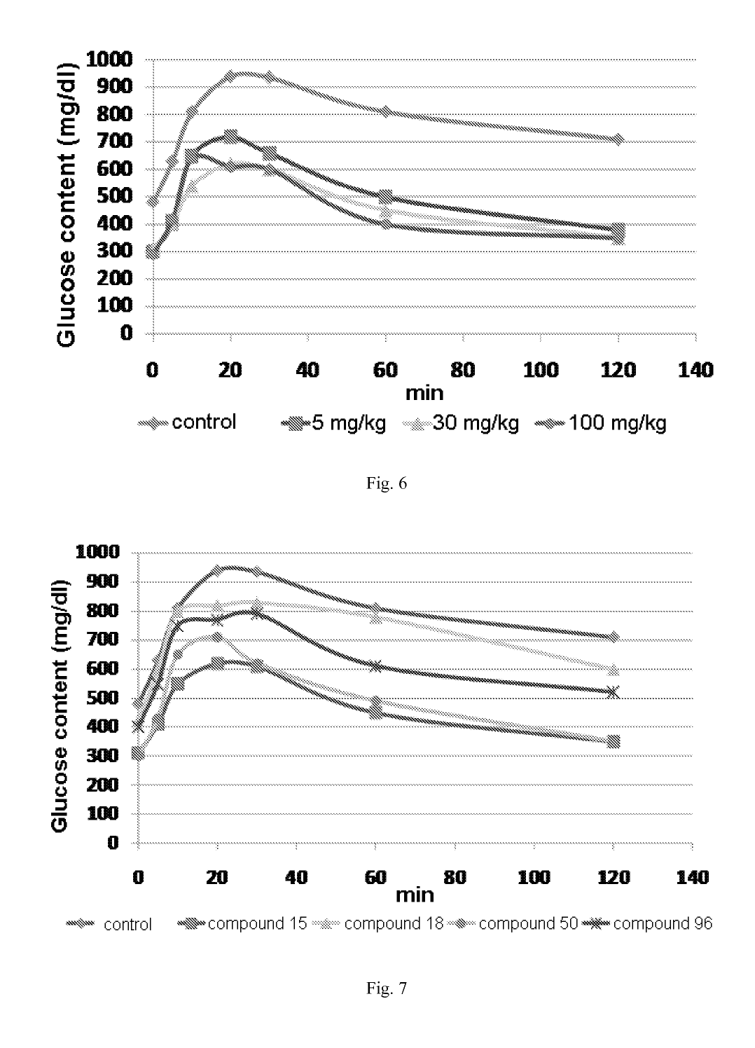

FIG. 6 Glucose concentration (mg/dl) in plasma of DIO-mice under the influence of agonist of TGR5 receptors of formula 15.

FIG. 7 Glucose concentration (mg/dl) in plasma of DIO-mice under the influence of agonist of TGR5 receptors of formulas 15, 18, 50, 96.

Below the invention is described by means of specific examples, which illustrate but not limit the scope of the invention.

Structures of the prepared compounds were confirmed by chemical, chromatographic and spectral analysis data.

### EXAMPLE 1

General method for preparation of compounds of the general formulas 1.1 and 1.3. The corresponding nitrile of the general formula A1 (1 eqv.) was reduced with hydrogen over Raney-nickel at room temperature and pressure of 2 atm. for 24 h. Then methanol was added, the catalyst was filtered off through celit, the solvent was evaporated in vacuo. The benzene solution of the obtained product was added dropwise during 1 h to a dispersion of LiAlH₄ (1.2 eqv.) in ether; after that the reaction mixture was refluxed for 24 h. As the reaction was completed, 10% K₂CO₃ solution was added dropwise, the mixture was heated to boiling and filtered. The filtrate was evaporated in vacuo, and the prepared compound of the general formula A2 was dissolved in chloroform, mixed with triethylamine (1.2 eqv.), cooled to 0° C., and 1.1 eqv. of the appropriate biaryl acid chloride was added. The obtained mixture was refluxed for 5 h, cooled to room temperature, and quenched with water. The organic layer was washed subsequently with 10% K₂CO₃ water solution and water, dried; the solvent was evaporated under reduced pressure. The structures of synthesized compounds were verified by LCMS data. Compounds 12-07, 11-13 were prepared in analogous manner.

02: MW 397.40; LCMS m/z 398(M+1);

03: MW 397.40; LCMS m/z 398(M+1); 04: MW 397.40; LCMS m/z 398(M+1);

05: MW 348.38; LCMS m/z 349(M+1); 06: MW 348.38; LCMS m/z 349(M+1);

07: MW 398.38; LCMS m/z 399(M+1); 11: MW 344.42; LCMS m/z 345(M+1);

12: MW 344.42; LCMS m/z 345(M+1); 13: MW 344.42; LCMS m/z 345(M+1);

and also the following compounds:
109: 4-(3-phenylbenzoyl)-2,3,4,5-tetrahydropyrido[3,4-f][1,4]oxazepine, MW 330.39; LCMS m/z 331 (M+1);
110: 4-(4-phenylbenzoyl)-2,3,4,5-tetrahydropyrido[3,4-f][1,4]oxazepine, MW 330.39; LCMS m/z 331 (M+1);
111: 4-(2-phenylbenzoyl)-2,3,4,5-tetrahydro pyrido[3,4-f][1,4]oxazepine, MW 330.39; LCMS m/z 331 (M+1).

### EXAMPLE 2

### General Method for Preparation of Some Compounds Presented by the General Formula 1

### Structure B2

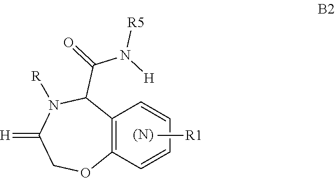

wherein R, R1 and R5 have the above meanings.

The corresponding aldehyde-acid of the general formula B1 (1.0 eq.) and primary biaryl amine (1.0 eq.) were dissolved in MeOH and stirred at 20° C. for 10 min. Then suitable isonitrile (1.2 eq.) was added and the resultant mixture was heated at 50° C. for 1-5 hs at stirring. After the reaction was completed the reaction mixture was cooled till 20° C., the solid precipitated was filtered of and washed with methanol. The prepared compound was recrystallized from ester or purified by chromatograpy on SiO₂ using as eluent CH₂Cl₂ with appropriate methanol gradient. Structures of compounds were confirmed by LCMS data.

Using this procedure compounds 81-87, 89-101 were prepared:

81: Molecular weight (M.w.) 508.55, LCMS m/z 509 (M+1);

82: M.w. 522.57; LCMS m/z 523 (M+1); 83: M.w. 496.53; LCMS m/z 497 (M+1);

84: M.w. 447.51; LCMS m/z 448 (M+1); 85: M.w. 447.51; LCMS m/z 448 (M+1);

86: M.w. 458.56; LCMS m/z 459 (M+1); 87: M.w. 459.55; LCMS m/z 460 (M+1);

89: M.w. 472.55; LCMS m/z 473 (M+1); 90: M.w. 429.52; LCMS m/z 430 (M+1);

91: M.w. 429.52; LCMS m/z 430 (M+1); 92: M.w. 429.52; LCMS m/z 430 (M+1);

93: M.w. 458.56; LCMS m/z 459 (M+1); 94: M.w. 458.56; LCMS m/z 459 (M+1);

95: M.w. 458.56; LCMS m/z 459 (M+1); 96: M.w. 448.56; LCMS m/z 449 (M+1);

97: M.w. 448.56; LCMS m/z 449 (M+1); 98: M.w. 448.56; LCMS m/z 449 (M+1);

99: M.w. 446.53; LCMS m/z 447 (M+1); 100: M.w. 446.53; LCMS m/z 447 (M+1);

101: M.w. 446.53; LCMS m/z 447 (M+1);

and also the following compounds:
168: 4-(3-phenylbenzyl)-3-oxo-2,3,4,5-tetrahydropyrido[3,4-f][1,4]oxazepine-5-carboxylic acid cyclopentylamide, M.w. 441.53; LCMS m/z 442 (M+1);
169: 4-(3-phenylbenzyl)-3-oxo-2,3,4,5-tetrahydropyrido[3,4-f][1,4]oxazepine-5-carboxylic acid cyclohexylamide, M.w. 455.56; LCMS m/z 456 (M+1);
170: 4-(2-phenylbenzyl)-3-oxo-2,3,4,5-tetrahydropyrido[3,4-f][1,4]oxazepine-5-carboxylic acid tert-butylamide, M.w. 429.52; LCMS m/z 430 (M+1);
171: 4-[2-(pyridin-4-yl)benzyl]-3-oxo-2,3,4,5-tetrahydropyrido[3,4-f][1,4]oxazepine-5-carboxylic acid tert-butylamide, M.w. 430.51; LCMS m/z 431 (M+1);
172: 4-[2-(pyridin-2-yl)benzyl]-3-oxo-2,3,4,5-tetrahydropyrido[3,4-f][1,4]oxazepine-5-carboxylic acid tert-butylamide, M.w. 430.51; LCMS m/z 431 (M+1);
173: 4-[2-(3,4-methylenedioxyphenyl)benzyl]-3-oxo-2,3,4,5-tetrahydropyrido[3,4-f][1,4]oxazepine-5-carboxylic acid tert-butylamide, M.w. 473.53; LCMS m/z 474 (M+1);
174: 4-[2-(pyridin-3-yl)benzyl)]-3-oxo-2,3,4,5-tetrahydropyrido[3,4-f][1,4]oxazepine-5-carboxylic acid tert-butylamide, M.w. 430.51; LCMS m/z 431 (M+1);
175: 4-[2-(4-methoxyphenyl)benzyl]-3-oxo-2,3,4,5-tetrahydropyrido[3,4-f][1,4]oxazepine-5-carboxylic acid tert-butylamide, M.w. 459.55; LCMS m/z 460 (M+1);
176: 4-[2-(3-methoxyphenyl)benzyl]-3-oxo-2,3,4,5-tetrahydropyrido[3,4-f][1,4]oxazepine-5-carboxylic acid tert-butylamide, M.w. 459.55; LCMS m/z 460 (M+1);
177: 4-[2-(2-methoxyphenyl)benzyl]-3-oxo-2,3,4,5-tetrahydropyrido[3,4-f][1,4]oxazepine-5-carboxylic acid tert-butylamide, M.w. 459.55; LCMS m/z 460 (M+1);
178: 4-[2-(4-methylphenyl)benzyl]-3-oxo-2,3,4,5-tetrahydropyrido[3,4-f][1,4]oxazepine-5-carboxylic acid tert-butylamide, M.w. 443.55; LCMS m/z 444 (M+1);
179: 4-[2-(3-methylphenyl)benzyl]-3-oxo-2,3,4,5-tetrahydropyrido[3,4-f][1,4]oxazepine-5-carboxylic acid tert-butylamide, M.w. 443.55; LCMS m/z 444 (M+1);
180: 4-[2-(2-methylphenyl)benzyl]-3-oxo-2,3,4,5-tetrahydropyrido[3,4-f][1,4]oxazepine-5-carboxylic acid tert-butylamide, M.w. 443.55; LCMS m/z 444 (M+1);
181: 4-[2-(4-fluoro phenyl)benzyl]-3-oxo-2,3,4,5-tetrahydropyrido[3,4-f][1,4]oxazepine-5-carboxylic acid tert-butylamide, M.w. 447.51; LCMS m/z 448 (M+1);
182: 4-[2-(3-fluoro phenyl)benzyl]-3-oxo-2,3,4,5-tetrahydropyrido[3,4-f][1,4]oxazepine-5-carboxylic acid tert-butylamide, M.w. 447.51; LCMS m/z 448 (M+1);
183: 4-[2-(2-fluorophenyl)benzyl]-3-oxo-2,3,4,5-tetrahydropyrido[3,4-f][1,4]oxazepine-5-carboxylic acid tert-butylamide, M.w. 447.51; LCMS m/z 448 (M+1);
244: 6-(3-phenylbenzyl)-3-oxo-5,6,7,8-tetrahydropyrimido[5,4-f][1,4]oxazepine-5-carboxylic acid cyclopentylamide, M.w. 442.52; LCMS m/z 443 (M+1);
245: 6-(3-phenylbenzyl)-3-oxo-5,6,7,8-tetrahydropyrimido[5,4-f][1,4]oxazepine-5-carboxylic acid cyclohexylamide, M.w. 456.55; LCMS m/z 457 (M+1);
246: 6-(2-phenylbenzyl)-3-oxo-5,6,7,8-tetrahydropyrimido[5,4-f][1,4]oxazepine-5-carboxylic acid tert-butylamide, M.w. 430.51; LCMS m/z 431 (M+1);
247: 6-[2-(pyridin-4-yl)benzyl]-3-oxo-5,6,7,8-tetrahydropyrimido[5,4-f][1,4]oxazepine-5-carboxylic acid tert-butylamide, M.w. 431.50; LCMS m/z 432 (M+1);
248: 6-[2-(pyridin-2-yl)benzyl]-3-oxo-5,6,7,8-tetrahydropyrimido[5,4-f][1,4]oxazepine-5-carboxylic acid tert-butylamide, M.w. 431.50; LCMS m/z 432 (M+1);
249: 6-[2-(3,4-methylenedioxyphenyl)benzyl]-3-oxo-5,6,7,8-tetrahydropyrimido[5,4-f][1,4]oxazepine-5-carboxylic acid tert-butylamide, M.w. 474.52; LCMS m/z 475 (M+1);
250: 6-[2-(pyridin-3-yl)benzyl]-3-oxo-5,6,7,8-tetrahydropyrimido[5,4-f][1,4]oxazepine-5-carboxylic acid tert-butylamide, M.w. 431.50; LCMS m/z 432 (M+1);
251: 6-[2-(4-methoxyphenyl)benzyl]-3-oxo-5,6,7,8-tetrahydropyrimido[5,4-f][1,4]oxazepine-5-carboxylic acid tert-butylamide, M.w. 460.54; LCMS m/z 461 (M+1);
252: 6-[2-(3-methoxyphenyl)benzyl]-3-oxo-5,6,7,8-tetrahydropyrimido[5,4-f][1,4]oxazepine-5-carboxylic acid tert-butylamide, M.w. 460.54; LCMS m/z 461 (M+1);
253: 6-[2-(2-methoxyphenyl)benzyl]-3-oxo-5,6,7,8-tetrahydropyrimido[5,4-f][1,4]oxazepine-5-carboxylic acid tert-butylamide, M.w. 460.54; LCMS m/z 461 (M+1);
254: 6-[2-(4-methylphenyl)benzyl]-3-oxo-5,6,7,8-tetrahydropyrimido[5,4-f][1,4]oxazepine-5-carboxylic acid tert-butylamide, M.w. 444.54; LCMS m/z 445 (M+1);
255: 6-[2-(3-methylphenyl)benzyl]-3-oxo-5,6,7,8-tetrahydropyrimido[5,4-f][1,4]oxazepine-5-carboxylic acid tert-butylamide, M.w. 444.54; LCMS m/z 445 (M+1);
256: 6-[2-(2-methylphenyl)benzyl]-3-oxo-5,6,7,8-tetrahydropyrimido[5,4-f][1,4]oxazepine-5-carboxylic acid tert-butylamide, M.w. 444.54; LCMS m/z 445 (M+1);
257: 6-[2-(4-fluorophenyl)benzyl]-3-oxo-5,6,7,8-tetrahydropyrimido[5,4-f][1,4]oxazepine-5-carboxylic acid tert-butylamide, M.w. 448.50; LCMS m/z 449 (M+1);
258: 6-[2-(3-fluorophenyl)benzyl]-3-oxo-5,6,7,8-tetrahydropyrimido[5,4-f][1,4]oxazepine-5-carboxylic acid tert-butylamide, M.w. 448.50; LCMS m/z 449 (M+1);
259: 6-[2-(2-fluorophenyl)benzyl]-3-oxo-5,6,7,8-tetrahydropyrimido[5,4-f][1,4]oxazepine-5-carboxylic acid tert-butylamide, M.w. 448.50; LCMS m/z 449 (M+1).

### EXAMPLE 3

General Method for Preparation Compounds of the General Formulas 1.2 and 1.4.

A solution of compound of the general formula B2 (1.0 mmol) in anhydrous THF (3 ml) was treated with a 2.0M solution of borane dimethylsulfide complex (2.0 ml, 4.0 mmol) in THF. The mixture was stirred at 20° C. for 10-12 hs, after that the solvent was evaporated, the residue was dissolved in saturated HCl solution in MeOH. The obtained solution was refluxed for 30 min, cooled, neutralized with 10% K₂CO₃ water solution and extracted with CH₂Cl₂ (3×50 ml). Combined organic extracts were dried over MgSO₄ and evaporated in vacuo. The residue was purified either by preparative TLC (SiO₂, eluent CH₂Cl₂), or HPLC method. Structures of compounds were confirmed by LCMS data.

Using this procedure, compounds 14, 15, 17-19, 24-26, 50-52 were prepared:

14: M.w. 494.56; LCMS m/z 495 (M+1); 15: M.w. 508.59; LCMS m/z 509 (M+1);

17: M.w. 482.55; LCMS m/z 483 (M+1); 18: M.w. 433.53; LCMS m/z 434 (M+1);

19: M.w. 433.53; LCMS m/z 434 (M+1); 24: M.w. 415.54; LCMS m/z 416 (M+1);

25: M.w. 415.54; LCMS m/z 416 (M+1); 26: M.w. 415.54; LCMS m/z 416 (M+1);

50: M.w. 450.53; LCMS m/z 451 (M+1); 51: M.w. 450.53; LCMS m/z 451 (M+1);

52: M.w. 450.53; LCMS m/z 451 (M+1);

and also compounds:
102: 4-[2-(4-dimethylaminophenyl)benzyl]-2,3,4,5-tetrahydrobenzo[f][1,4]oxazepine-5-carboxylic acid, M.w. 402.49; LCMS m/z 403 (M+1);
103: 4-[2-(4-diethylaminophenyl)benzyl]-2,3,4,5-tetrahydrobenzo[f][1,4]oxazepine-5-carboxylic acid, M.w. 430.55; LCMS m/z 431 (M+1);
104: 4-(2-phenylbenzyl)-7-methoxy-2,3,4,5-tetrahydrobenzo[f][1,4]oxazepine-5-carboxylic acid phenylamide, M.w. 464.56; LCMS m/z 465 (M+1);
105: 4-(2-phenylbenzyl]-7-methoxy-2,3,4,5-tetrahydrobenzo[f][1,4]oxazepine-5-carboxylic acid m-pyridinylamide, M.w. 465.56; LCMS m/z 466 (M+1);
106: 4-[2-(4-cyanophenyl)benzyl]-2,3,4,5-tetrahydrobenzo[f][1,4]oxazepine, M.w. 340.42; LCMS m/z 341 (M+1);
107: 4-(2-phenylbenzyl]-7-methoxy-2,3,4,5-tetrahydrobenzo[f][1,4]oxazepine-5-carboxylic acid benzylamide, M.w. 478.59; LCMS m/z 479 (M+1);
113: 4-[(4-phenylpyridin-3-yl)methyl]-2,3,4,5-tetrahydropyrido[3,4-f][1,4]oxazepine, M.w. 317.39; LCMS m/z 318 (M+1);
114: 4-[2-(pyridin-4-yl)benzyl]-2,3,4,5-tetrahydropyrido[3,4-f][1,4]oxazepine, M.w. 317.39; LCMS m/z 318 (M+1);
115: 4-[2-(pyridin-2-yl)benzyl]-2,3,4,5-tetrahydropyrido[3,4-f][1,4]oxazepine, M.w. 317.39; LCMS m/z 318 (M+1);
118: 4-(3-phenylbenzyl)-2,3,4,5-tetrahydropyrido[3,4-f][1,4]oxazepine-5-carboxylic acid cyclopentylamide, M.w. 427.55; LCMS m/z 428 (M+1);
119: 4-(3-phenylbenzyl)-2,3,4,5-tetrahydropyrido[3,4-f][1,4]oxazepine-5-carboxylic acid cyclohexylamide, M.w. 441.58; LCMS m/z 442 (M+1);
120: 4-(4-phenylbenzyl)-2,3,4,5-tetrahydropyrido[3,4-f][1,4]oxazepine-5-carboxylic acid, M.w. 360.42; LCMS m/z 361 (M+1);
121: 4-(2-phenylbenzyl)-2,3,4,5-tetrahydropyrido[3,4-f][1,4]oxazepine-5-carboxylic acid tert-butylamide, M.w. 415.54; LCMS m/z 416 (M+1);
122: 4-[2-(pyridin-4-yl)benzyl]-2,3,4,5-tetrahydropyrido[3,4-f][1,4]oxazepine-5-carboxylic acid tert-butylamide, M.w. 416.53; LCMS m/z 417 (M+1);
123: 4-[2-(pyridin-2-yl)benzyl]-2,3,4,5-tetrahydropyrido[3,4-f][1,4]oxazepine-5-carboxylic acid tert-butylamide, M.w. 416.53; LCMS m/z 417 (M+1);
125: 4-[2-(pyridin-3-yl)benzyl]-2,3,4,5-tetrahydropyrido[3,4-f][1,4]oxazepine-5-carboxylic acid tert-butylamide, M.w. 416.53; LCMS m/z 417 (M+1);
127: 4-[2-(pyridin-4-yl)benzyl]-2,3,4,5-tetrahydropyrido[3,4-f][1,4]oxazepine-5-carboxylic acid, M.w. 361.40; LCMS m/z 362 (M+1);
128: 4-[2-(pyridin-2-yl)benzyl]-2,3,4,5-tetrahydropyrido[3,4-f][1,4]oxazepine-5-carboxylic acid, M.w. 361.40; LCMS m/z 362 (M+1);
129: 4-[2-(pyridin-3-yl)benzyl]-2,3,4,5-tetrahydropyrido[3,4-f][1,4]oxazepine-5-carboxylic acid, M.w. 361.40; LCMS m/z 362 (M+1);
131: 4-[2-(pyridin-4-yl)benzyl]-2,3,4,5-tetrahydropyrido[3,4-f][1,4]oxazepine-5-carboxylic acid ethyl ester, M.w. 389.46; LCMS m/z 390 (M+1);
132: 4-[2-(pyridin-2-yl)benzyl]-2,3,4,5-tetrahydropyrido[3,4-f][1,4]oxazepine-5-carboxylic acid ethyl ester, M.w. 389.46; LCMS m/z 390 (M+1);
133: 4-[2-(pyridin-3-yl)benzyl]-2,3,4,5-tetrahydropyrido[3,4-f][1,4]oxazepine-5-carboxylic acid ethyl ester, M.w. 389.46; LCMS m/z 390 (M+1);
134: 4-[2-(4-methoxyphenyl)benzyl]-2,3,4,5-tetrahydropyrido[3,4-f][1,4]oxazepine-5-carboxylic acid tert-butylamide, M.w. 445.57; LCMS m/z 446 (M+1);
135: 4-[2-(3-methoxyphenyl)benzyl]-2,3,4,5-tetrahydropyrido[3,4-f][1,4]oxazepine-5-carboxylic acid tert-butylamide, M.w. 445.57; LCMS m/z 446 (M+1);
136: 4-[2-(2-methoxyphenyl)benzyl]-2,3,4,5-tetrahydropyrido[3,4-f][1,4]oxazepine-5-carboxylic acid tert-butylamide, M.w. 445.57; LCMS m/z 446 (M+1);
137: 4-[2-(4-methylphenyl)benzyl]-2,3,4,5-tetrahydropyrido[3,4-f][1,4]oxazepine-5-carboxylic acid tert-butylamide, M.w. 429.57; LCMS m/z 430 (M+1);
138: 4-[2-(3-methylphenyl)benzyl]-2,3,4,5-tetrahydropyrido[3,4-f][1,4]oxazepine-5-carboxylic acid tert-butylamide, M.w. 429.57; LCMS m/z 430 (M+1);
139: 4-[2-(2-methylphenyl)benzyl]-2,3,4,5-tetrahydropyrido[3,4-f][1,4]oxazepine-5-carboxylic acid tert-butylamide, M.w. 429.57; LCMS m/z 430 (M+1);
140: 4-[2-(4-fluorophenyl)benzyl]-2,3,4,5-tetrahydropyrido[3,4-f][1,4]oxazepine-5-carboxylic acid tert-butylamide, M.w. 433.53; LCMS m/z 434 (M+1);
141: 4-[2-(3-fluorophenyl)benzyl]-2,3,4,5-tetrahydropyrido[3,4-f][1,4]oxazepine-5-carboxylic acid tert-butylamide, M.w. 433.53; LCMS m/z 434 (M+1);
142: 4-[2-(2-fluorophenyl)benzyl]-2,3,4,5-tetrahydropyrido[3,4-f][1,4]oxazepine-5-carboxylic acid tert-butylamide, M.w. 433.53; LCMS m/z 434 (M+1);
143: 4-[2-(4-chlorophenyl)benzyl]-2,3,4,5-tetrahydropyrido[3,4-f][1,4]oxazepine-5-carboxylic acid tert-butylamide, M.w. 449.98; LCMS m/z 450 (M+1);
144: 4-[2-(3-chlorophenyl)benzyl]-2,3,4,5-tetrahydropyrido[3,4-f][1,4]oxazepine-5-carboxylic acid tert-butylamide, M.w. 449.98; LCMS m/z 450 (M+1);
145: 4-[2-(2-chlorophenyl)benzyl]-2,3,4,5-tetrahydropyrido[3,4-f][1,4]oxazepine-5-carboxylic acid tert-butylamide, M.w. 449.98; LCMS m/z 450 (M+1);
146: 4-[2-(pyrimidin-4-yl)benzyl]-2,3,4,5-tetrahydropyrido[3,4-f][1,4]oxazepine-5-carboxylic acid tert-butylamide, M.w. 417.52; LCMS m/z 418 (M+1);
147: 4-[2-(pyrimidin-2-yl)benzyl]-2,3,4,5-tetrahydropyrido[3,4-f][1,4]oxazepine-5-carboxylic acid tert-butylamide, M.w. 417.52; LCMS m/z 418 (M+1);
148: 4-[2-(pyrimidin-5-yl)benzyl]-2,3,4,5-tetrahydropyrido[3,4-f][1,4]oxazepine-5-carboxylic acid tert-butylamide, M.w. 417.52; LCMS m/z 418 (M+1);
149: 4-[2-(pyrazin-2-yl)benzyl]-2,3,4,5-tetrahydropyrido[3,4-f][1,4]oxazepine-5-carboxylic acid tert-butylamide, M.w. 417.52; LCMS m/z 418 (M+1);
150: 4-[2-(pyrazin-2-yl)benzyl]-2,3,4,5-tetrahydropyrido[3,4-f][1,4]oxazepine-5-carboxylic acid cyclopentylamide, M.w. 429.53; LCMS m/z 430 (M+1);
166: 4-[2-(2-chlorophenyl)benzyl]-2,3,4,5-tetrahydropyrido[3,4-f][1,4]oxazepine-5-carboxylic acid, M.w. 394.86; LCMS m/z 395 (M+1);
167: 4-(2-phenylbenzyl)-2,3,4,5-tetrahydropyrido[3,4-f][1,4]oxazepine-5-carboxylic acid, M.w. 360.42; LCMS m/z 361 (M+1);
184: 6-(3-phenyl)-5,6,7,8-tetrahydropyrimido[5,4-f][1,4]oxazepine, M.w. 317.39; LCMS m/z 318 (M+1);
189: 6-[(4-phenylpyridin-3-yl)methyl]-5,6,7,8-tetrahydropyrimido[5,4-f][1,4]oxazepine, M.w. 318.38; LCMS m/z 319 (M+1);
190: 6-[2-(pyridin-4-yl)benzyl]-5,6,7,8-tetrahydropyrimido[5,4-f][1,4]oxazepine, M.w. 318.38; LCMS m/z 319 (M+1);
191: 6-[2-(pyridin-2-yl)benzyl]-5,6,7,8-tetrahydropyrimido[5,4-f][1,4]oxazepine, M.w. 318.38; LCMS m/z 319 (M+1);
194: 6-(3-phenylbenzyl)-5,6,7,8-tetrahydropyrimido[5,4-f][1,4]oxazepine-5-carboxylic acid cyclopentylamide, M.w. 428.54; LCMS m/z 429 (M+1);
195: 6-(3-phenylbenzyl)-5,6,7,8-tetrahydropyrimido[5,4-f][1,4]oxazepine-5-carboxylic acid cyclohexylamide, M.w. 442.57; LCMS m/z 443 (M+1);
196: 6-(4-phenylbenzyl)-5,6,7,8-tetrahydropyrimido[5,4-f][1,4]oxazepine-5-carboxylic acid, M.w. 361.40; LCMS m/z 362 (M+1);
197: 6-(2-phenylbenzyl)-5,6,7,8-tetrahydropyrimido[5,4-f][1,4]oxazepine-5-carboxylic acid tert-butylamide, M.w. 416.53; LCMS m/z 417 (M+1);
198: 6-[2-(pyridin-4-yl)benzyl]-5,6,7,8-tetrahydropyrimido[5,4-f][1,4]oxazepine-5-carboxylic acid tert-butylamide, M.w. 417.52; LCMS m/z 418 (M+1);
199: 6-[2-(pyridin-2-yl)benzyl]-5,6,7,8-tetrahydropyrimido[5,4-f][1,4]oxazepine-5-carboxylic acid tert-butylamide, M.w. 417.52; LCMS m/z 418 (M+1);
201: 6-[2-(pyridin-3-yl)benzyl]-5,6,7,8-tetrahydropyrimido[5,4-f][1,4]oxazepine-5-carboxylic acid tert-butylamide, M.w. 417.52; LCMS m/z 418 (M+1);
203: 6-[2-(pyridin-4-yl)benzyl]-5,6,7,8-tetrahydropyrimido[5,4-f][1,4]oxazepine-5-carboxylic acid, M.w. 362.39; LCMS m/z 363 (M+1);
204: 6-[2-(pyridin-2-yl)benzyl]-5,6,7,8-tetrahydropyrimido[5,4-f][1,4]oxazepine-5-carboxylic acid, M.w. 362.39; LCMS m/z 363 (M+1);
205: 6-[2-(pyridin-3-yl)benzyl]-5,6,7,8-tetrahydropyrimido[5,4-f][1,4]oxazepine-5-carboxylic acid, M.w. 362.39; LCMS m/z 363 (M+1);
207: 6-[2-(pyridin-4-yl)benzyl]-5,6,7,8-tetrahydropyrimido[5,4-f][1,4]oxazepine-5-carboxylic acid ethyl ester, M.w. 390.45; LCMS m/z 391 (M+1);
208: 6-[2-(pyridin-2-yl)benzyl]-5,6,7,8-tetrahydropyrimido[5,4-f][1,4]oxazepine-5-carboxylic acid ethyl ester, M.w. 390.45; LCMS m/z 391 (M+1);
209: 6-[2-(pyridin-3-yl)benzyl]-5,6,7,8-tetrahydropyrimido[5,4-f][1,4]oxazepine-5-carboxylic ethyl ester, M.w. 390.45; LCMS m/z 391 (M+1);
210: 6-[2-(4-methoxyphenyl)benzyl]-5,6,7,8-tetrahydropyrimido[5,4-f][1,4]oxazepine-5-carboxylic acid tert-butylamide, M.w. 446.55; LCMS m/z 447 (M+1);
211: 6-[2-(3-methoxyphenyl)benzyl]-5,6,7,8-tetrahydropyrimido[5,4-f][1,4]oxazepine-5-carboxylic acid tert-butylamide, M.w. 446.55; LCMS m/z 447 (M+1);
212: 6-[2-(2-methoxyphenyl)benzyl]-5,6,7,8-tetrahydropyrimido[5,4-f][1,4]oxazepine-5-carboxylic acid tert-butylamide, M.w. 446.55; LCMS m/z 447 (M+1);
213: 6-[2-(4-methylphenyl)benzyl]-5,6,7,8-tetrahydropyrimido[5,4-f][1,4]oxazepine-5-carboxylic acid tert-butylamide, M.w. 430.55; LCMS m/z 431 (M+1);
214: 6-[2-(3-methylphenyl)benzyl]-5,6,7,8-tetrahydropyrimido[5,4-f][1,4]oxazepine-5-carboxylic acid tert-butylamide, M.w. 430.55; LCMS m/z 431 (M+1);
215: 6-[2-(2-methylphenyl)benzyl]-5,6,7,8-tetrahydropyrimido[5,4-f][1,4]oxazepine-5-carboxylic acid tert-butylamide, M.w. 430.55; LCMS m/z 431 (M+1);
216: 6-[2-(4-fluorophenyl)benzyl]-5,6,7,8-tetrahydropyrimido[5,4-f][1,4]oxazepine-5-carboxylic acid tert-butylamide, M.w. 434.52; LCMS m/z 435 (M+1);
217: 6-[2-(3-fluorophenyl)benzyl]-5,6,7,8-tetrahydropyrimido[5,4-f][1,4]oxazepine-5-carboxylic acid tert-butylamide, M.w. 434.52; LCMS m/z 435 (M+1);
218: 6-[2-(2-fluorophenyl)benzyl]-5,6,7,8-tetrahydropyrimido[5,4-f][1,4]oxazepine-5-carboxylic acid tert-butylamide, M.w. 434.52; LCMS m/z 435 (M+1);
219: 6-[2-(4-chlorophenyl)benzyl]-5,6,7,8-tetrahydropyrimido[5,4-f][1,4]oxazepine-5-carboxylic acid tert-butylamide, M.w. 450.97; LCMS m/z 451 (M+1);
220: 6-[2-(3-chlorophenyl)benzyl]-5,6,7,8-tetrahydropyrimido[5,4-f][1,4]oxazepine-5-carboxylic acid tert-butylamide, M.w. 450.97; LCMS m/z 451 (M+1);
221: 6-[2-(2-chlorophenyl)benzyl]-5,6,7,8-tetrahydropyrimido[5,4-f][1,4]oxazepine-5-carboxylic acid tert-butylamide, M.w. 450.97; LCMS m/z 451 (M+1);
222: 6-[2-(pyrimidin-4-yl)benzyl]-5,6,7,8-tetrahydropyrimido[5,4-f][1,4]oxazepine-5-carboxylic acid tert-butylamide, M.w. 418.50; LCMS m/z 419 (M+1);
223: 6-[2-(pyrimidin-2-yl)benzyl]-5,6,7,8-tetrahydropyrimido[5,4-f][1,4]oxazepine-5-carboxylic acid tert-butylamide, M.w. 418.50; LCMS m/z 419 (M+1);
224: 6-[2-(pyrimidin-5-yl)benzyl]-5,6,7,8-tetrahydropyrimido[5,4-f][1,4]oxazepine-5-carboxylic acid tert-butylamide, M.w. 418.50; LCMS m/z 419 (M+1);
225: 6-[2-(pyrazin-2-yl)benzyl]-5,6,7,8-tetrahydropyrimido[5,4-f][1,4]oxazepine-5-carboxylic acid tert-butylamide, M.w. 418.50; LCMS m/z 419 (M+1);
226: 6-[2-(pyrazin-2-yl)benzyl]-5,6,7,8-tetrahydropyrimido[5,4-f][1,4]oxazepine-5-carboxylic acid cyclopentylamide, M.w. 430.51; LCMS m/z 431 (M+1);
242: 6-[2-(2-chlorophenyl)benzyl]-5,6,7,8-tetrahydropyrimido[5,4-f][1,4]oxazepine-5-carboxylic acid, M.w. 395.85; LCMS m/z 396 (M+1);
243: 6-(2-phenylbenzyl)-5,6,7,8-tetrahydropyrimido[5,4-f][1,4]oxazepine-5-carboxylic acid, M.w. 361.40; LCMS m/z 362 (M+1).

### EXAMPLE 4

Determination of Agonistic Activity of Some of the Compounds of the General Formula 1 Towards TGR5 Receptors

Screening of compounds of the general formula 1 was carried out in experiments on cells of HEK-293 line, in which human TGR5 (hTGR5) receptors were expressed. Agonistic activity of compounds was estimated by increasing of intracellular cAMP concentration. For the most active compounds EC50 values (concentration of compound in μM at which 50% of maximal effect is achieved) were determined from concentration dependences. Table 1 represents data for some compounds of the general formula 1, at that, A means, that EC₅₀<1.0 mcmol; B—1.0 mcmol<EC₅₀<10 mcmol, C—EC₅₀>10 mcmol.

TABLE 1
Agonistic activity of some of the
compounds of the general formula 1.

| Compound | Activity |
|---|---|
| 14 | C |
| 15 | A |
| 18 | B |
| 50 | A |
| 81 | C |
| 90 | C |
| 96 | B |
| 97 | C |

### EXAMPLE 5

### Influence of Rectal Administration of Bile Acids (BA)

The influence of rectal administration of glycodeoxycholic acid (GDC) on glucose level in blood was studied at diabesity mice of db/db line. Glucose level at male db/db mice (39.5 g) was measured 24 hs before and at the moment of rectal administration, and then in 1, 2, 3, 4, 5, 6, and 24 hs afterwards. The mice received rectally four compositions: 1) gel without any active components (vehicle only); 2) gel without any active components, accompanied, however, with rectal administration of DPP4 inhibitor Sitagliptin (vehicle+Sitagliptin); 3) gel comprising GDC (5 mmol) and accompanied with peroral administration of DPP4 inhibitor Sitagliptin (GDC 5 mmol+Sitagliptin); 4) gel comprising GDC (50 mmol) and accompanied with peroral administration of DPP4 inhibitor Sitagliptin (GDC 50 mmol+Sitagliptin). Sitagliptin inhibitor was administered perorally in dose of 30 mg/kg 15 min before rectal administration of compositions. The gel was prepared on the base of methyl cellulose water solution (1%) and Tween-80 (2%). GDC BA administration in both doses effectively and statistically valid reduced glucose concentration in plasma of diabesity mice of db/db line (on 24%—at 50 mmol and on 20% at 5 mmol). Administration of DPP4 inhibitor Sitagliptin alone lowered glucose level in blood plasma on 5-10% only.

These findings are definitely indicative of positive effect of the proposed method for treatment of metabolic diseases.

### EXAMPLE 6

### Influence of Rectal BA Administration on Weight Change at Diabesity

Influence of rectal administration of glycodeoxycholic acid (GDC) on weight change of mice of db/db line at diabesity was investigated. The mice received rectally four compositions: 1) gel without any active components (vehicle only); 2) gel without any active components, accompanied, however, with rectal administration of DPP4 inhibitor Sitagliptin (vehicle+Sitagliptin); 3) gel comprising GDC (5 mmol) and accompanied with peroral administration of DPP4 inhibitor Sitagliptin (GDC 5 mmol+Sitagliptin); 4) gel comprising GDC (50 mmol) and accompanied with peroral administration of DPP4 inhibitor Sitagliptin (GDC 50 mmol+Sitagliptin). Sitagliptin inhibitor was administered peroral in dose of 30 mg/kg 15 min before rectal administration of compositions. The gel was prepared on the base of methyl cellulose water solution (1%) and Tween-80 (2%). The animals were weighted 2 days before the experiment, at the moment of administration and 2 days after administration of compositions. GDC BA administration in both doses effectively and statistically-valid reduced weights of experimental animals.

These results are definitely indicative of effective weight lowering of tested animals.

### EXAMPLE 7

### Modelling of Metabolic Syndrome at Human Using DIO-Mice

DIO-mice (Diet Induced Obesity mice—mice with diet induced obesity) are widely used for modelling of metabolic syndrome at human, which is characterized by abdominal obesity, high level of triglycerides, glucose homeostasis disturbance and hyperinsulinemia [Hariri N, Thibault L. High-fat diet-induced obesity in animal models. Nutr. Res. Rev. 2010; 23(2): 270-99]. For the development of DIO-model C57BL/6J mice were used, which had been on high fat diet (high fat diet HFD; nutriment includes 58% of lard. Diet D12492, Research Diet, New Brunswick, N.J.; West D., et al. 1992). HFD-diet and water were given to the animals ad libitum for 28 days before the experiment. The DIO-model obtained was used for investigation of farmacological effect of compounds of the general formula 1 on secretion of GLP-1 peptide and glucose metabolism.

### EXAMPLE 8

### Study of Pharmacological Effect of Compounds of the General Formula 1 on Secretion of Incretin GLP-1 Peptide

The tested compound 4-(3-phenylbenzyl)-7-trifluoromethyl-2,3,4,5-tetrahydrobenzo[f][1,4]oxazepine-5-carboxylic acid cyclihexylamide (15) of the general formula 1 (dose 30 mg/kg) in water solution of carboxymethyl cellulose (0.5%) and Tween-20 (0.25%) was administered perorally to DIO-mice C57BL/6J (6 mice for a timepoint) 15 min before peroral administration of dextrose (2 g/kg in physiological solution). Blood for analysis was taken from the tail vein of animals in 5, 10, 15 and 30 min after sugar administration. Water solution of carboxymethyl cellulose (0.5%) and Tween-20 (0.25%) was administered as a control. The test was repeated for compounds 18, 50 and 96 of the general formula 1.

The content of incretin GLP-1 peptide in blood plasma was determined by standard ELISA method. FIG. 4 shows stimulation of GLP-1 secretion in 15 and 30 min after administration of sugar (or in 30 and 45 min after administration of the compound). In all cases administration of compounds of the general formula 1 (30 mg/kg) effectively and statistically-valid stimulates GLP-1 hormone stimulation.

### EXAMPLE 9

### Investigation of Combined Action of Compounds of the General Formula 1 and DPP4 Sitagliptin Inhibitor

As it follows from the data shown in FIG. 4, the content of GLP-1 peptide in plasma markedly decreases because of its degradation under the action of proteases, mainly DPP4 proteinase (in all cases GLP-1 concentration decreases in 30 min in comparison with the data for 15 min).

Therefore, we studied the influence of DPP4 proteinase inhibitor on the stability of GLP-1 peptide in plasma, secretion of which was induced by the action of TGR5 receptors agonist in the presence of glucose. Sitagliptin inhibitor was administered peroral in dose of 30 mg/kg 25 min before administration of sugar. As follows from the data shown in FIG. 5, DPP4 proteinase inhibitor maintains high GLP-1 concentration in plasma that increases effectiveness of TGR5 agonist application.

These results uniquely testify positive effect of the proposed method for treatment of metabolic diseases.

### EXAMPLE 10

### Study of Pharmacological Action of Compounds of the General Formula 1 on Glucose Metabolism (Glucose Tolerance Test GTT)

The tested compound 4-(3-phenylbenzyl)-7-trifluoromethyl-2,3,4,5-tetrahydrobenzo[f][1,4]oxazepine-5-carboxylic acid cyclohexylamide (15) of the general formula 1 (doses from 5 to 100 mg/kg) in water solution of carboxymethyl cellulose (0.5%) and Tween-20 (0.25%) was administered peroral to DIO-mice C57BL/6J (6 mice for a time point) 15 min before peroral administration of dextrose (2 g/kg in physiological solution). Blood for analysis was taken from the tail vein of animals in 0, 5, 10, 20, 30, 60 and 120 min after sugar administration. Water solution of carboxymethyl cellulose (0.5%) and Tween-20 (0.25%) was administered as a control. The test was repeated for compounds 18, 50 and 96 of the general formula 1.

Data shown in FIGS. 6 and 7 indicate a significant statistically valid lowering of glucose concentration in blood plasma of DIO-mice in response to the action of TGR5 receptor agonists.

These results clearly indicate perspectiveness of the proposed approach to the treatment of metabolic diseases.

### INDUSTRIAL APPLICABILITY

The invention could be used in medicine, veterinary, biochemistry.

1. A compound of general formula 1, or a pharmaceutically acceptable salt thereof, 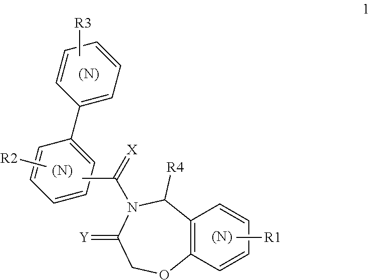
wherein:
R1, R2 and R3 independently of each other are hydrogen, a C₁-C₃ alkyl, halogen, a trifluoromethyl, a C₁-C₃ alkoxy, a cyano group, a trifluoromethoxy group; an amino group substituted with C₁-C₃ alkyl; or
two radicals R3 at the adjacent carbon atoms of the benzene ring, together with this benzene ring form 3,4-methylenedioxyphenyl;
R4 is hydrogen, a C₁-C₅ alkyl, a carboxyl group, a C₁-C₃ alkoxycarbonyl or an amide group CONHR5; wherein R5 is an optionally substituted C₁-C₃ alkyl, a C₅-C₆ cycloalkyl, an optionally substituted phenyl, benzyl, pyridyl;
X and Y are two hydrogen atoms or an oxygen, provided that
Y═O and X=2H, or Y=2H and X═O, or X═Y=2H;
(N)—denotes that benzene ring could be bioisosterically replaced by azaheterocyclic ring such as: pyridine, pyrimidine, pyridazine, triazine or pyrazine.

2. The compound of claim 1 of formula 1.1 or 1.2, or a pharmaceutically acceptable salt thereof, 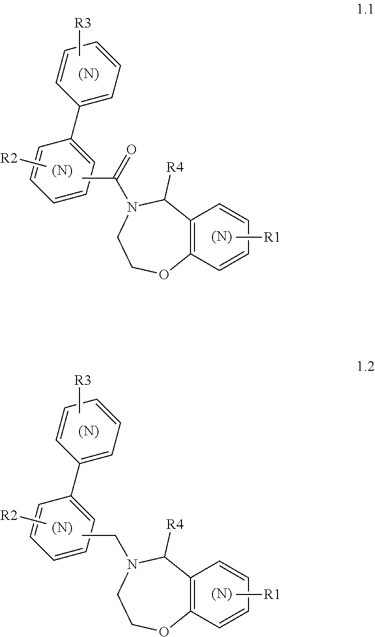
wherein: R1, R2, R3, R4 and (N) are said above.

3. The compound of claim 2 of formula 1.3 or 1.4 or a pharmaceutically acceptable salt thereof, 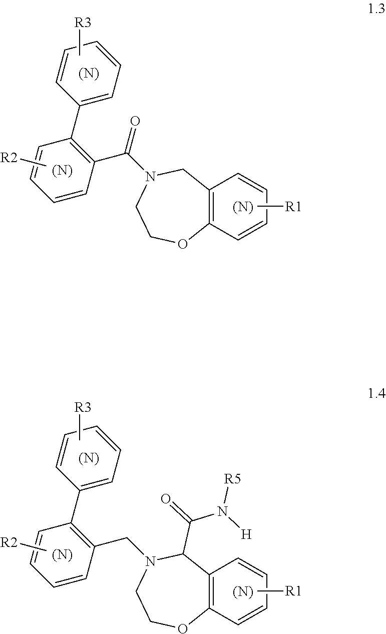
wherein R1, R2, R3, R5 and (N) are said above.

4. The compound of claim 1 selected from the group consisting of:
4-(3-phenylbenzyl)-7-trifluoromethyl-2,3,4,5-tetrahydrobenzo[f][1,4]oxazepine (01),
4-(3-phenylbenzoyl)-7-trifluoromethyl-2,3,4,5-tetrahydrobenzo[f][1,4]oxazepine (02),
4-(4-phenylbenzoyl)-7-trifluoromethyl-2,3,4,5-tetrahydrobenzo[f][1,4]oxazepine (03),
4-(2-phenylbenzoyl)-7-trifluoromethyl-2,3,4,5-tetrahydrobenzo[f][1,4]oxazepine (04),
4-[2-(pyridin-4-yl)benzoyl]-7-fluoro-2,3,4,5-tetrahydrobenzo[f][1,4]oxazepine (05),
4-[2-(pyridin-2-yl)benzoyl]-7-fluoro-2,3,4,5-tetrahydrobenzo[f][1,4]oxazepine (06),
4-(4-phenylnicotinoyl)-7-trifluoromethyl-2,3,4,5-tetrahydrobenzo[f][1,4]oxazepine (07),
4-[(4-phenylpyridin-3-yl)methyl]-7-trifluoromethyl-2,3,4,5-tetrahydrobenzo[f][1,4]oxazepine (08),
4-[2-(pyridin-4-yl)benzyl]-7-fluoro-2,3,4,5-tetrahydrobenzo[f][1,4]oxazepine (09),
4-[2-(pyridin-2-yl)benzyl]-7-fluoro-2,3,4,5-tetrahydrobenzo[f][1,4]oxazepine (10),
4-(4-phenylnicotinoyl)-7-methyl-2,3,4,5-tetrahydrobenzo[f][1,4]oxazepine (11),
4-[2-(pyridin-4-yl)benzoyl]-7-methyl-2,3,4,5-tetrahydrobenzo[f][1,4]oxazepine (12),
4-[2-(pyridin-2-yl)benzoyl]-7-methyl-2,3,4,5-tetrahydrobenzo[f][1,4]oxazepine (13),
4-(3-pheylbenzyl)-7-trifluoromethyl-2,3,4,5-tetrahydrobenzo[f][1,4]oxazepine-5-carboxylic acid cyclopentylamide (14),
4-(3-phenylbenzyl)-7-trifluoromethyl-2,3,4,5-tetrahydrobenzo[f][1,4]oxazepine-5-carboxylic acid cyclohexylamide (15),
4-(4-phenylbenzyl)-7-trifluoromethyl-2,3,4,5-tetrahydrobenzo[f][1,4]oxazepine-5-carboxylic acid (16),
4-(2-phenylbenzyl)-7-trifluoromethyl-2,3,4,5-tetrahydrobenzo[f][1,4]oxazepine-5-carboxylic acid tert-butylamide (17),
4-[2-(pyridin-4-yl)benzyl]-7-fluoro-2,3,4,5-tetrahydrobenzo[f][1,4]oxazepine-5-carboxylic acid tert-butylamide (18),
4-[2-(pyridin-2-yl)benzyl]-7-fluoro-2,3,4,5-tetrahydrobenzo[f][1,4]oxazepine-5-carboxylic acid tert-butylamide (19),
4-(2-phenylbenzyl)-7-methoxy-2,3,4,5-tetrahydrobenzo[f][1,4]oxazepine-5-carboxylic acid tert-butylamide (20),
4-[2-(pyridin-4-yl)benzyl]-7-methoxy-2,3,4,5-tetrahydrobenzo[f][1,4]oxazepine-5-carboxylic acid tert-butylamide (21),
4-[2-(pyridin-2-yl)benzyl]-7-methoxy-2,3,4,5-tetrahydrobenzo[f][1,4]oxazepine-5-carboxylic acid tert-butylamide (22),
4-[2-(3,4-methylenedioxyphenyl)benzyl]-2,3,4,5-tetrahydrobenzo[f][1,4]oxazepine-5-carboxylic acid tert-butylamide (23),
4-[2-(pyridin-4-yl)benzyl]-2,3,4,5-tetrahydrobenzo[f][1,4]oxazepine-5-carboxylic acid tert-butylamide (24),
4-[2-(pyridin-2-yl)benzyl]-2,3,4,5-tetrahydrobenzo[f][1,4]oxazepine-5-carboxylic acid tert-butylamide (25),
4-[2-(pyridin-3-yl)benzyl]-2,3,4,5-tetrahydrobenzo[f][1,4]oxazepine-5-carboxylic acid tert-butylamide (26),
4-[2-(3,4-methylenedioxyphenyl)benzyl]-2,3,4,5-tetrahydrobenzo[f][1,4]oxazepine-5-carboxylic acid (27),
4-[2-(pyridin-4-yl)benzyl]-2,3,4,5-tetrahydrobenzo[f][1,4]oxazepine-5-carboxylic acid (28),
4-[2-(pyridin-2-yl)benzyl]-2,3,4,5-tetrahydrobenzo[f][1,4]oxazepine-5-carboxylic acid (29),
4-[2-(pyridin-3-yl)benzyl]-2,3,4,5-tetrahydrobenzo[f][1,4]oxazepine-5-carboxylic acid (30),
4-[2-(3,4-methylenedioxyphenyl)benzyl]-2,3,4,5-tetrahydrobenzo[f][1,4]oxazepine-5-carboxylic acid ethyl ester (31),
4-[2-(pyridin-4-yl)benzyl]-2,3,4,5-tetrahydrobenzo[f][1,4]oxazepine-5-carboxylic acid ethyl ester (32),
4-[2-(pyridin-2-yl)benzyl]-2,3,4,5-tetrahydrobenzo[f][1,4]oxazepine-5-carboxylic acid ethyl ester (33),
4-[2-(pyridin-3-yl)benzyl]-2,3,4,5-tetrahydrobenzo[f][1,4]oxazepine-5-carboxylic acid ethyl ester (34),
4-[2-(4-methoxyphenyl)benzyl]-2,3,4,5-tetrahydrobenzo[f][1,4]oxazepine-5-carboxylic acid tert-butylamide (35),
4-[2-(3-methoxyphenyl)benzyl]-2,3,4,5-tetrahydrobenzo[f][1,4]oxazepine-5-carboxylic acid tert-butylamide (36),
4-[2-(2-methoxyphenyl)benzyl]-2,3,4,5-tetrahydrobenzo[f][1,4]oxazepine-5-carboxylic acid tert-butylamide (37),
4-[2-(4-methylphenyl)benzyl]-2,3,4,5-tetrahydrobenzo[f][1,4]oxazepine-5-carboxylic acid tert-butylamide (38),
4-[2-(3-methylphenyl)benzyl]-2,3,4,5-tetrahydrobenzo[f][1,4]oxazepine-5-carboxylic acid tert-butylamide (39),
4-[2-(2-methylphenyl)benzyl]-2,3,4,5-tetrahydrobenzo[f][1,4]oxazepine-5-carboxylic acid tert-butylamide (40),
4-[2-(4-fluorophenyl)benzyl]-2,3,4,5-tetrahydrobenzo[f][1,4]oxazepine-5-carboxylic acid tert-butylamide (41),
4-[2-(3-fluorophenyl)benzyl]-2,3,4,5-tetrahydrobenzo[f][1,4]oxazepine-5-carboxylic acid tert-butylamide (42),
4-[2-(2-fluorophenyl)benzyl]-2,3,4,5-tetrahydrobenzo[f][1,4]oxazepine-5-carboxylic acid tert-butylamide (43),
4-[2-(4-chlorophenyl)benzyl]-2,3,4,5-tetrahydrobenzo[f][1,4]oxazepine-5-carboxylic acid tert-butylamide (44),
4-[2-(3-chlorophenyl)benzyl]-2,3,4,5-tetrahydrobenzo[f][1,4]oxazepine-5-carboxylic acid tert-butylamide (45),
4-[2-(2-chlorophenyl)benzyl]-2,3,4,5-tetrahydrobenzo[f][1,4]oxazepine-5-carboxylic acid tert-butylamide (46),
4-[2-(4-methylphenyl)benzyl]-7-fluoro-2,3,4,5-tetrahydrobenzo[f][1,4]oxazepine-5-carboxylic acid tert-butylamide (47),
4-[2-(3-methylphenyl)benzyl]-7-fluoro-2,3,4,5-tetrahydrobenzo[f][1,4]oxazepine-5-carboxylic acid tert-butylamide (48),
4-[2-(2-methylphenyl)benzyl]-7-fluoro-2,3,4,5-tetrahydrobenzo[f][1,4]oxazepine-5-carboxylic acid tert-butylamide (49),
4-[2-(4-fluorophenyl)benzyl]-7-fluoro-2,3,4,5-tetrahydrobenzo[f][1,4]oxazepine-5-carboxylic acid tert-butylamide (50),
4-[2-(3-fluorophenyl)benzyl]-7-fluoro-2,3,4,5-tetrahydrobenzo[f][1,4]oxazepine-5-carboxylic acid tert-butylamide (51),
4-[2-(2-fluorophenyl)benzyl]-7-fluoro-2,3,4,5-tetrahydrobenzo[f][1,4]oxazepine-5-carboxylic acid tert-butylamide (52),
4-[2-(pyrimidin-4-yl)benzyl]-2,3,4,5-tetrahydrobenzo[f][1,4]oxazepine-5-carboxylic acid tert-butylamide (53),
4-[2-(pyrimidin-2-yl)benzyl]-2,3,4,5-tetrahydrobenzo[f][1,4]oxazepine-5-carboxylic acid tert-butylamide (54),
4-[2-(pyrimidin-5-yl)benzyl]-2,3,4,5-tetrahydrobenzo[f][1,4]oxazepine-5-carboxylic acid tert-butylamide (55),
4-[2-(pyrazin-2-yl)benzyl]-2,3,4,5-tetrahydrobenzo[f][1,4]oxazepine-5-carboxylic acid tert-butylamide (56),
4-[2-(pyrazin-2-yl)benzyl]-2,3,4,5-tetrahydrobenzo[f][1,4]oxazepine-5-carboxylic acid cyclopentylamide (57),
4-[2-(pyrazin-2-yl)benzyl]-7-fluoro-2,3,4,5-tetrahydrobenzo[f][1,4]oxazepine-5-carboxylic acid cyclopentylamide (58),
4-[2-(pyrazin-2-yl)benzyl]-7-fluoro-2,3,4,5-tetrahydrobenzo[f][1,4]oxazepine-5-carboxylic acid (59),
4-[2-(pyrimidin-4-yl)benzyl]-2,3,4,5-tetrahydrobenzo[f][1,4]oxazepine-5-carboxylic acid (60),
4-[2-(pyrimidin-2-yl)benzyl]-2,3,4,5-tetrahydrobenzo[f][1,4]oxazepine-5-carboxylic acid (61),
4-[2-(pyrimidin-5-yl)benzyl]-2,3,4,5-tetrahydrobenzo[f][1,4]oxazepine-5-carboxylic acid (62),
4-[2-(4-methoxyphenyl)benzyl]-2,3,4,5-tetrahydrobenzo[f][1,4]oxazepine-5-carboxylic acid (63),
4-[2-(3-methoxyphenyl)benzyl]-2,3,4,5-tetrahydrobenzo[f][1,4]oxazepine-5-carboxylic acid (64),
4-[2-(2-methoxyphenyl)benzyl]-2,3,4,5-tetrahydrobenzo[f][1,4]oxazepine-5-carboxylic acid (65),
4-[2-(4-methylphenyl)benzyl]-2,3,4,5-tetrahydrobenzo[f][1,4]oxazepine-5-carboxylic acid (66),
4-[2-(3-methylphenyl)benzyl]-2,3,4,5-tetrahydrobenzo[f][1,4]oxazepine-5-carboxylic acid (67),
4-[2-(2-methylphenyl)benzyl]-2,3,4,5-tetrahydrobenzo[f][1,4]oxazepine-5-carboxylic acid (68),
4-[2-(4-fluorophenyl)benzyl]-2,3,4,5-tetrahydrobenzo[f][1,4]oxazepine-5-carboxylic acid (69),
4-[2-(3-fluorophenyl)benzyl]-2,3,4,5-tetrahydrobenzo[f][1,4]oxazepine-5-carboxylic acid (70),
4-[2-(2-fluorophenyl)benzyl]-2,3,4,5-tetrahydrobenzo[f][1,4]oxazepine-5-carboxylic acid (71),
4-[2-(4-chlorophenyl)benzyl]-2,3,4,5-tetrahydrobenzo[f][1,4]oxazepine-5-carboxylic acid (72),
4-[2-(3-chlorophenyl)benzyl]-2,3,4,5-tetrahydrobenzo[f][1,4]oxazepine-5-carboxylic acid (73),
4-[2-(2-chlorophenyl)benzyl]-2,3,4,5-tetrahydrobenzo[f][1,4]oxazepine-5-carboxylic acid (74),
4-[2-(4-fluorophenyl)benzyl]-7-fluoro-2,3,4,5-tetrahydrobenzo[f][1,4]oxazepine-5-carboxylic acid (75),
4-[2-(3-fluorophenyl)benzyl]-7-fluoro-2,3,4,5-tetrahydrobenzo[f][1,4]oxazepine-5-carboxylic acid (76),
4-[2-(2-fluorophenyl)benzyl]-7-fluoro-2,3,4,5-tetrahydrobenzo[f][1,4]oxazepine-5-carboxylic acid (77),
4-(2-phenylbenzyl)-7-trifluoromethyl-2,3,4,5-tetrahydrobenzo[f][1,4]oxazepine-5-carboxylic acid (78),
4-(2-phenylbenzyl)-7-trifluoromethoxy-2,3,4,5-tetrahydrobenzo[f][1,4]oxazepine-5-carboxylic acid (79),
4-(2-phenylbenzyl)-7-tert-butyl-2,3,4,5-tetrahydrobenzo[f][1,4]oxazepine-5-carboxylic acid (80),
4-(3-phenylbenzyl)-3-oxo-7-trifluoromethyl-2,3,4,5-tetrahydrobenzo[f][1,4]oxazepine-5-carboxylic acid cyclopentylamide (81),
4-(3-phenylbenzyl)-3-oxo-7-trifluoromethyl-2,3,4,5-tetrahydrobenzo[f][1,4]oxazepine-5-carboxylic acid cyclohexylamide (82),
4-(2-phenylbenzyl)-3-oxo-7-trifluoromethyl-2,3,4,5-tetrahydrobenzo[f][1,4]oxazepine-5-carboxylic acid tert-butylamide (83),
4-[2-(pyridin-4-yl)benzyl]-3-oxo-7-fluoro-2,3,4,5-tetrahydrobenzo[f][1,4]oxazepine-5-carboxylic acid tert-butylamide (84),
4-[2-(pyridin-2-yl)benzyl]-3-oxo-7-fluoro-2,3,4,5-tetrahydrobenzo[f][1,4]oxazepine-5-carboxylic acid tert-butylamide (85),
4-(2-phenylbenzyl)-3-oxo-7-methoxy-2,3,4,5-tetrahydrobenzo[f][1,4]oxazepine-5-carboxylic acid tert-butylamide (86),
4-[2-(pyridin-4-yl)benzyl]-3-oxo-7-methoxy-2,3,4,5-tetrahydrobenzo[f][1,4]oxazepine-5-carboxylic acid tert-butylamide (87),
4-[2-(pyridin-2-yl)benzyl]-3-oxo-7-methoxy-2,3,4,5-tetrahydrobenzo[f][1,4]oxazepine-5-carboxylic acid tert-butylamide (88),
4-[2-(3,4-methylenedioxyphenyl)benzyl]-3-oxo-2,3,4,5-tetrahydrobenzo[f][1,4]oxazepine-5-carboxylic acid tert-butylamide (89),
4-[2-(pyridin-4-yl)benzyl]-3-oxo-2,3,4,5-tetrahydrobenzo[f][1,4]oxazepine-5-carboxylic acid tert-butylamide (90),
4-[2-(pyridin-2-yl)benzyl]-3-oxo-2,3,4,5-tetrahydrobenzo[f][1,4]oxazepine-5-carboxylic acid tert-butylamide (91),
4-[2-(pyridin-3-yl)benzyl]-3-oxo-2,3,4,5-tetrahydrobenzo[f][1,4]oxazepine-5-carboxylic acid tert-butylamide (92),
4-[2-(4-methoxyphenyl)benzyl]-3-oxo-2,3,4,5-tetrahydrobenzo[f][1,4]oxazepine-5-carboxylic acid tert-butylamide (93),
4-[2-(3-methoxyphenyl)benzyl]-3-oxo-2,3,4,5-tetrahydrobenzo[f][1,4]oxazepine-5-carboxylic acid tert-butylamide (94),
4-[2-(2-methoxyphenyl)benzyl]-3-oxo-2,3,4,5-tetrahydrobenzo[f][1,4]oxazepine-5-carboxylic acid tert-butylamide (95),
4-[2-(4-methylphenyl)benzyl]-3-oxo-2,3,4,5-tetrahydrobenzo[f][1,4]oxazepine-5-carboxylic acid tert-butylamide (96),
4-[2-(3-methylphenyl)benzyl]-3-oxo-2,3,4,5-tetrahydrobenzo[f][1,4]oxazepine-5-carboxylic acid tert-butylamide (97),
4-[2-(2-methylphenyl)benzyl]-3-oxo-2,3,4,5-tetrahydrobenzo[f][1,4]oxazepine-5-carboxylic acid tert-butylamide (98),
4-[2-(4-fluorophenyl)benzyl]-3-oxo-2,3,4,5-tetrahydrobenzo[f][1,4]oxazepine-5-carboxylic acid tert-butylamide (99),
4-[2-(3-fluorophenyl)benzyl]-3-oxo-2,3,4,5-tetrahydrobenzo[f][1,4]oxazepine-5-carboxylic acid tert-butylamide (100), and
4-[2-(2-fluorophenyl)benzyl]-3-oxo-2,3,4,5-tetrahydrobenzo[f][1,4]oxazepine-5-carboxylic acid tert-butylamide (101).

5. A method for the preparation of a compound of formula 1.3 according to claim 3 comprising (i) reducing an o-cyanooxyacetate compound of formula A1 and subsequently cyclizing to form a compound of formula A2, (ii) acylating a compound of formula A2 and subsequently isolating or separating the reaction product, 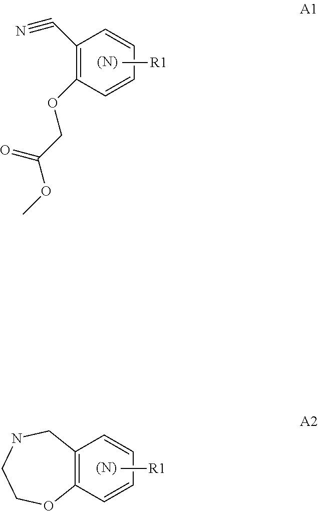
wherein R1, and (N) are said above.

6. A method for the preparation of a compound of formula 1.4 according to claim 3 comprising (i) interacting an aldehydro-acid compound of formula B1 with isonitrile and a suitable amine to form a compound of formula B2, (ii) reducing a compound of formula B2 and subsequently isolating or separating the reaction product, 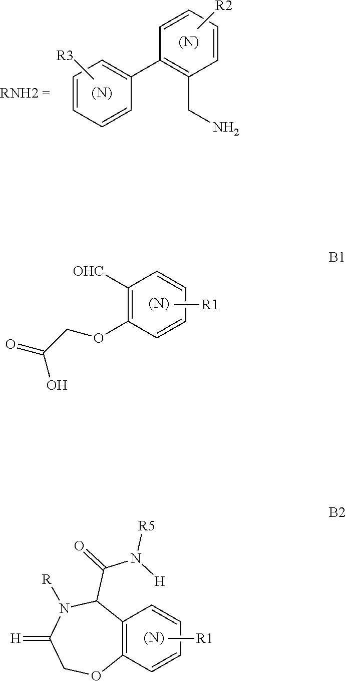
wherein R1, R5, and (N) are said above.

7. An active component comprising a compound of formula 1 according to claim 1.

8. A pharmaceutical composition comprising an active component of claim 7 in a pharmaceutically effective amount and at least one pharmaceutically acceptable excipient.

9. The pharmaceutical composition of claim 8 further comprising a proteinase DPP-IV inhibitor, selected from Vildagliptin or Sitagliptin.

10. The pharmaceutical composition according to any of claims 8, 9 in the form of a tablet, a capsule or an injection placed in a pharmaceutically acceptable package.

11. A combined medicament for prophylaxis and treatment of metabolic diseases, associated with glucose metabolism, comprising an active component according to claim 7 or a pharmaceutical composition according to any of claims 8-10 in an effective amount and a secretion stimulator selected from an endogenous bile acid or a mixture of endogenous bile acids in the form of a rectal suspension or a gel.

12. A method for treating a metabolic disease associated with glucose metabolism, comprising administering to a subject an active component according to claim 7 or a pharmaceutical composition according to any of claims 8-10 in need thereof.

13. A method for treating a metabolic disease associated with glucose metabolism, comprising administering to a subject a combined medicament according to claim 11, wherein a proteinase DPP-IV inhibitor is being peroral administered or in the form of an injection and an endogenous bile acid or a mixture of endogenous bile acids is being administered through rectum in need thereof.
